(19) United States
(12) Patent Application Publication
(10) Pub. No.: US 2022/0205030 A1
(43) Pub. Date: Jun. 30, 2022

(54) VACCINE-INDUCED GENE SIGNATURES CORRELATING WITH PROTECTION AGAINST HIV AND SIV INFECTION

(71) Applicant: THE HENRY M. JACKSON FOUNDATION FOR THE ADVANCEMENT OF MILITARY MEDICINE, INC., BETHESDA, MD (US)

(72) Inventor: Rasmi THOMAS, Silver Spring, MD (US)

(21) Appl. No.: 17/429,698

(22) PCT Filed: Feb. 11, 2020
(86) PCT No.: PCT/US2020/017617

§ 371 Date: Aug. 10, 2021

Related U.S. Application Data
(60) Provisional application No. 62/803,724, filed on Feb. 11, 2019.

(51) Int. Cl. C12Q 1/6851; C12Q 1/6876

(52) U.S. Cl. CPC C12Q 1/6851; C12Q 2600/158; C12Q 1/6876

(57) ABSTRACT

Provided are methods for predicting effectiveness of vaccine against HIV or SIV. Provided herein also are methods for predicting protective immunity in a subject, comprising detecting expression levels of multiple genes or gene products, wherein the multiple genes have been identified as a differentially expressed gene set associated with protective immunity against HIV or SIV; calculating a first composite gene expression score (GES) for the differentially expressed gene set; and calculating an average second composite GES for the differentially expressed gene set in biological samples from subjects that have been vaccinated with a candidate vaccine and are not immune to the virus, wherein a first composite GES that is greater than the average second composite GES indicates the effectiveness of the vaccine and that the subject has protective immunity. Also provided are kits for predicting effectiveness of HIV or SIV vaccine and for predicting protective immunity following vaccination.

### CROSS-REFERENCE TO RELATED APPLICATION

This application claims the benefit of, and relies on the filing date of, U.S. provisional patent application No. 62/803,724, filed 11 Feb. 2019, the entire disclosure of which is incorporated herein by reference.

### GOVERNMENT INTEREST

This invention was made in part with Government support under grant number W81XWH-11-2-0174 awarded by the United States Army. The Government has certain rights in the invention.

### SEQUENCE LISTING

The present application contains a Sequence Listing which has been submitted electronically in ASCII format and is hereby incorporated by reference in its entirety. Said ASCII copy, created on 10 Feb. 2020, is named HMJ-164-PCT_SL.txt and is 656,835 bytes in size.

### FIELD

This application generally relates to human immunodeficiency virus (HIV) and simian immunodeficiency virus (SIV) vaccines and vaccine candidates.

### BACKGROUND

An estimated 1.8 million new cases of HIV infection were diagnosed worldwide in 2017, and approximately 36.9 million people are currently living with AIDS/HIV. Although AIDS-related deaths have been dramatically reduced in recent years, an estimated 940,000 people nonetheless died from AIDS-related complications worldwide in 2017, and there remains no cure.

Attempts at an efficacious preventative HIV vaccine for humans have been elusive to date. In one study, an HIV-1 vaccine showed partial efficacy in people from Thailand, although that efficacy was insufficient for development and subsequent use [S. Rerks-Ngarm et al., Vaccination with ALVAC and AIDSVAX to prevent HIV-1 infection in Thailand, The New England Journal of Medicine 361, 2209-2220 (2009)]. The study contained a combination of two vaccines (an ALVAC® prime and an AIDSVAX® boost) based on HIV strains common in Thailand. Although the efficacy of the study vaccine trial was only about 31%, mathematical modeling has demonstrated that with effective pre-exposure prophylaxis, the ability to reduce HIV risk by even 50 percent could be an effective public health approach to the HIV pandemic [J. Medlock et al., Effectiveness of UNAIDS targets and HIV vaccination across 127 countries, Proc. Natl. Acad. Sci. USA 2017 Apr. 11; 114(15):4017-4022].

Some vaccine regimens have shown at least this level of efficacy in non-human primate (NHP), SIV and Simian-Human Immunodeficiency Virus (SHIV) challenge models. In one study, for example, Barouch et al. identified a protective efficacy of greater than 50 percent in an SIV challenge study with an adenovirus serotype 26 (Ad26) prime, followed by purified envelope glycoprotein (gp140) boost vaccine regimen [D. H. Barouch et al., Protective efficacy of adenovirus/protein vaccines against SIV challenges in rhesus monkeys, Science 349, 320-324 (2015) (hereinafter “Barouch I”)]. These findings were replicated in an SHIV challenge model in an additional independent NHP study [D. H. Barouch et al., Evaluation of a mosaic HIV-1 vaccine in a multicentre, randomised, double-blind, placebo-controlled, phase 1/2a clinical trial (APPROACH) and in rhesus monkeys (NHP 13-19), Lancet 392, 232-243 (2018) (hereinafter “Barouch II”)].

Identification of gene signatures that are indicative of a protective immune response from acquiring HIV or SIV, such as gene signatures from the aforementioned studies, may be useful to predict immune responses of vaccinated subjects and to advance vaccine development.

### SUMMARY

One aspect of the present disclosure is directed to methods of predicting effectiveness of a human immunodeficiency virus (HIV) or a simian immunodeficiency virus (SIV) vaccine in a subject that has been vaccinated with the HIV or the SIV vaccine, the method comprising (a) detecting expression levels of multiple genes and/or gene products in a biological sample from the subject, wherein the multiple genes and/or gene products have been identified as a differentially expressed gene set associated with efficacy of the HIV or the SIV vaccine; (b) calculating a first composite gene expression score (GES) for the differentially expressed gene set in the biological sample from the subject; and (c) calculating an average second composite GES for the differentially expressed gene set in a plurality of biological samples from subjects that have been vaccinated with the HIV or SIV vaccine and are not immune to HIV or SIV, wherein a first composite GES that is greater than the average second composite GES indicates effectiveness of the HIV vaccine or the SIV infection. In certain embodiments, the method further comprises a step of identifying the differentially expressed gene set associated with efficacy of the HIV or the SIV vaccine. In certain embodiments, the method further comprises a step of adjusting treatment options for the subject based on the predicted effectiveness of the HIV or the SIV vaccine.

Another aspect of the present disclosure is directed to methods of predicting protective immunity to HIV or SIV infection in a subject that has been vaccinated with a HIV or SIV vaccine, the method comprising (a) detecting expression levels of multiple genes and/or gene products in a biological sample from the subject, wherein the multiple genes and/or gene products have been identified as a differentially expressed gene set associated with protective immunity against HIV or SIV; (b) calculating a first composite gene expression score (GES) for the differentially expressed gene set in the biological sample from the subject; and (c) calculating an average second composite GES for the differentially expressed gene set in biological samples from a plurality of subjects that have been vaccinated with the HIV or the SIV vaccine and are not immune to HIV or SIV, wherein a first composite GES that is greater than the average second composite GES indicates the subject that has been vaccinated with the HIV or the SIV vaccine has protective immunity to HIV or SIV infection. In certain embodiments, the method further comprises a step of identifying the differentially expressed gene set associated with protective immunity to HIV or SIV infection. In certain embodiments, the method further comprises a step of adjusting treatment options for the subject based on whether the subject is predicted to have protective immunity to HIV or SIV infection.

In some embodiments of all aspects of the present disclosure, the HIV or SIV vaccine comprises an adenovirus serotype 26 (Ad26) vector-based vaccine, a canarypox virus vector-based vaccine, or a retrovirus vector-based vaccine; in some embodiments of all aspects of the present disclosure, the HIV or SIV vaccine comprises an HIV or SIV envelope glycoprotein. In certain embodiments of all aspects of the disclosure, the subject that has been vaccinated with a HIV or SIV vaccine is a human, and in certain embodiments of all aspects of the disclosure, the subject is a non-human animal, such as a non-human primate.

In certain embodiments of all aspects of the disclosure, the HIV vaccine comprises a recombinant canarypox genetically engineered to express HIV-1 Gag and Pro (subtype B LAI strain) and CRF01_AE (subtype E) HIV-1 gp120 (92TH023) linked to the transmembrane 3 anchoring portion of gp41 (LAI). In some embodiments, the HIV vaccine comprises a bivalent HIV gp120 envelope glycoprotein vaccine containing a subtype E envelope from the HIV-1 strain A244 (CM244) and a subtype B envelope from the HIV-1 MN, optionally produced in Chinese hamster ovary cell lines.

In certain embodiments of all aspects of the disclosure, the differentially expressed gene set is associated with a B-cell gene signature or a monocyte gene signature. In some embodiments of all aspects of the disclosure, the biological sample is a blood sample, and in some embodiments of all aspects of the disclosure, the biological sample comprises peripheral blood mononuclear cells, such as lymphocytes, white blood cells, granulocytes, monocytes, macrophages, or any combination thereof.

In certain embodiments of all aspects of the disclosure, the first and second composite GES are calculated by averaging Z scores calculated by comparison to normalized gene expression for each gene in the differentially expressed gene set. In certain embodiments of all aspects of the disclosure, the normalized gene expression is measured from a biological sample of a subject or a pool of biological samples from subjects that have not been vaccinated with the HIV vaccine or the SIV vaccine and are not infected with HIV or SIV.

In certain embodiments of all aspects of the disclosure, the differentially expressed gene set comprises at least 3, at least 10, at least 15, at least 20, at least 25, at least 30, or 32 of the following genes: ACSL1, BHLHE40, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, OGFRL1, PHACTR2, RAB32, REEP4, RNF130, RRAGD, SDCBP, SIRPA, ST3GAL6, TKT, TNFRSF1B, TNFSF13, and VEGFA, and in certain embodiments of all aspects of the disclosure, the differentially expressed gene set comprises at least BHLHE40, OGFRL1, and TNFSF13.

In certain embodiments of all aspects of the disclosure, the differentially expressed gene set comprises at least 7, at least 10, at least 20, at least 30, at least 40, at least 50, at least 60, or 63 of the following genes: ADAM9, ALDH3B1, AMPD2, AP2S1, ARRB1, C5AR1, CAMKK2, CCR1, CD14, CD163, CD68, CEBPA, CSFIR, CST3, CTBP2, CTSD, DAPK1, DUSP6, FCGRT, FLVCR2, FZDJ, GAA, GAS7, GRN, HMOX1, IL17RA, IMPA2, KIAA0513, LEPROT, LGALS2, LMNA, LRPAP1, MAFB, MPP1, MYO1F, NAGA, NPL, PGD, PKM2, PSAP, PTGER2, PXN, PYGL, QPCT, RAB20, RBM47, RNF130, SCARB2, SEMA4A, SERINC5, SIRPA, SIRPB1, SLC36A1, SLCO3A1, SORT1, SULT1A2, TBC1D8, TBXAS1, TMP2, TKT, TYROBP, ZDHHC7, and ZNF467, and in certain embodiments of all aspects of the disclosure, the differentially expressed gene set comprises at least SEMA4A, SLC36A1, SERINC5, IL17RA, CTSD, CD68, and GAA.

In some embodiments of all aspects of the disclosure, the first composite GES is calculated based on measuring or detecting mRNA, cDNA, or protein products of the genes in the differentially expressed gene set in the biological sample. In other embodiments of all aspects of the disclosure, the second composite GES is calculated based on measuring or detecting mRNA, cDNA, or protein products of the genes in the differentially expressed gene set in the plurality of biological samples.

Another aspect of the present disclosure is directed to a kit for use in predicting efficacy and/or effectiveness of an HIV or SIV vaccine in a subject. In some embodiments, a kit is disclosed for predicting protective immunity to HIV or SIV infection in subjects that have received HIV or SIV vaccines, such as mammals that have received HIV or SIV vaccines. The kit comprises reagents for measuring or determining expression levels of a plurality of genes and/or gene products and instructions for how to use the kit. In certain embodiments, the kit comprises a plurality of probes for detecting BHLHE40, OGFRL1, and TNFSF13 and at least 5, at least 10, at least 15, at least 20, at least 25, or 29 of the following additional genes: ACSL1, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, PHACTR2, RAB32, REEP4, RNF130, RRAGD, SDCBP, SIRPA, ST3GAL6, TKT, TNFRSF1B, and VEGFA, wherein the plurality of probes contains probes for detecting no more than 500 different genes. In certain embodiments, the kit comprises a plurality of probes for detecting expression levels of genes and/or gene products selected from the group consisting of SEMA44A, SLC36A1, SERINC5, IL17RA, CTSD, CD68, GAA and at least 5, at least 10, at least 20, at least 30, at least 40, at least 50, or 56 genes selected from the group consisting of ADAM9, ALDH3B1, AMPD2, AP2S1, ARRB1, C5AR1, CAMKK2, CCR1, CD14, CD163, CEBPA, CSF1R, CST3, CTBP2, DAPK1, DUSP6, FCGRT, FLVCR2, FZD1, GAS7, GRN, HMOX1, IMPA2, KIAA0513, LEPROT, LGALS2, LMNA, LRPAP1, MAFB, MPP1, MYOIF, NAGA, NPL, PGD, PKM2, PSAP, PTGER2, PAN, PYGL, QPCT, RAB20, RBM47, RNF130, SCARB2, SIRPA, SIRPB1, SLCO3A1, SORT1, SULT1A2, TBC1D8, TBXAS1, TIMP2, TKT, TYROBP, ZDHHC7, and ZNF467.

In certain embodiments of the kits disclosed herein, the plurality of probes contains probes for detecting no more than 250, 100, 75, 63, 60, 50, 40, 32, 30, 25, 20, 15, 10, or 8 different genes. In certain embodiments, the plurality of probes is attached to the surface of an array. In certain embodiments, the plurality of probes may be detectably labeled.

### BRIEF DESCRIPTION OF THE DRAWINGS

The accompanying drawings, which are included to provide a further understanding of the disclosure, are incorporated in and constitute a part of this specification. The drawings illustrate embodiments of the disclosure and, together with the detailed description, serve to explain the principles of the disclosure. No attempt is made to show structural details of the disclosure in more detail than may be necessary for a fundamental understanding of the disclosure and various ways in which it may be practiced.

FIG. 1 is a plot showing an Area under Receiver Operating Characteristics (ROC) Curve (AUC) for the GES calculated from the Barouch I study samples.

FIG. 2 is a plot showing the Area under Receiver Operating Characteristics (ROC) Curves (AUC) for the GES calculated from the Barouch II study samples.

FIG. 3 is a Kaplan-Meier plot of the GES as a categorical variable showing the percentage of uninfected subjects after viral challenge over time for the Barouch I study samples with high (above mean) or low (below mean) GES.

FIG. 4 is a Kaplan-Meier plot of the GES as a categorical variable showing the percentage of uninfected subjects after viral challenge over time for the Barouch II study samples with high (above mean) or low (below mean) GES.

FIG. 5 is a scatter plot showing the composite GES calculated using 32 differentially expressed genes in the Barouch I study population (n=10) for protected and unprotected subjects.

FIG. 6 is a scatter plot showing the composite GES calculated using 52 differentially expressed genes in the Ad26/gp140 arm of the Barouch II study population (n=11) for protected and unprotected subjects.

FIG. 7 is a scatter plot showing the composite GES calculated using 62 differentially expressed genes in the Ad26/Ad26+gp140 arm of the Barouch II study population (n=12) for protected and unprotected subjects.

FIG. 8 is a scatter plot showing the composite GES is statistically significantly greater for the uninfected human subjects who received an HIV vaccine compared to the infected human subjects who received an HIV vaccine, as discussed in Example 2.

FIG. 9 is a Kaplan-Meier plot showing the probability of acquiring HIV-1 over a period of 36 months and showing that the probability is lower in individuals with higher GES, as discussed in Example 2.

FIG. 10 shows average percent vaccine efficacy, demonstrating that the vaccine efficacy is statistically significantly greater in individuals with a high GES compared to those with a low GES, as discussed in Example 2.

FIG. 11 shows specific genes in the signature are associated with protection from HIV acquisition. Panel A is a radar plot showing specific genes that are associated with a decreased time to infection in a RV144 human study (P<0.05, q<0.1), as discussed in Example 2. Panel B shows the average gene expression and percent gene expression in various cell lineages, as identified by single cell RNA-seq, demonstrating the genes as mostly expressed in cells from the myeloid lineage.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

### Definitions

In order that the present invention may be more readily understood, certain terms are first defined. Additional definitions are set forth throughout the detailed description.

Throughout this specification and the claims which follow, unless the context requires otherwise, the word “comprise.” and variations such as “comprises” and “comprising,” will be understood to imply the inclusion of a stated integer or step or group of integers or steps but not the exclusion of any other integer or step or group of integers or steps. When used herein, the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “detecting” or “detection” means any of a variety of methods known in the art for determining the presence, absence, or amount of a nucleic acid or a protein. As used throughout the specification, the term “detecting” or “detection” includes either qualitative or quantitative detection.

The term “gene expression” refers to the expression level of a gene in a sample. As is understood in the art, the expression level of a gene can be analyzed by measuring the expression of a nucleic acid (e.g., mRNA or cDNA) or a polypeptide that is encoded by the nucleic acid.

The term “gene expression score” (GES) refers to a numerical value calculated by the Z score or an average of the Z scores of a gene or gene set in a sample or collection of samples relative to the normalized gene expression for the particular gene or gene set. As used herein, the Z score refers to the number of standard deviations above or below the mean, wherein the mean refers to the average gene expression score for a gene in all samples. The average of the Z scores for all genes across a sample set is equal to the GES. For example, in certain embodiments, the Z score may be calculated using the formula z=(X−μ)/std(X), wherein X is the gene expression for a particular gene in a sample, μ is the mean expression score of the gene for all samples, and std(X) is the standard deviation for the mean expression score.

As used herein, a “biological sample” comprises human or animal cells or nucleic acids or polypeptides isolated therefrom. A biological sample of the present disclosure includes, but is not limited to, a tissue sample, a cell sample, a blood sample, a serum sample, a semen or seminal fluid sample, a urine sample, a saliva sample, or any combination thereof.

The term “gene signature” refers to one or more genes or groups of genes having a characteristic pattern of expression that occurs as a result of a condition, such as immunity to a virus. The term is also used interchangeably in this application with “gene set” or “differentially expressed gene set.”

The term “isolated,” when used in the context of a polypeptide or nucleic acid refers to a polypeptide or nucleic acid that is substantially free of its natural environment and is thus distinguishable from a polypeptide or nucleic acid that might happen to occur naturally. For instance, an isolated polypeptide or nucleic acid is substantially free of cellular material or other polypeptides or nucleic acids from the cell or tissue source from which it was derived.

The term “normalized gene expression” refers to an average gene expression level for a given gene in a pool of samples that are free of the disease or virus.

The terms “polypeptide,” “peptide,” and “protein” are used interchangeably herein to refer to polymers of amino acids.

The term “polypeptide probe” as used herein refers to a labeled (e.g., fluorescently or isotopically labeled) polypeptide that can be used in a protein detection assay (e.g., mass spectrometry) to quantify a polypeptide of interest in a biological sample.

The term “primer” means a polynucleotide capable of binding to a region of a target nucleic acid, or its complement, and promoting nucleic acid amplification of the target nucleic acid. Generally, a primer will have a free 3′ end that can be extended by a nucleic acid polymerase. Primers also generally include a base sequence capable of hybridizing via complementary base interactions either directly with at least one strand of the target nucleic acid or with a strand that is complementary to the target sequence. A primer may comprise target-specific sequences and optionally other sequences that are non-complementary to the target sequence. These non-complementary sequences may comprise, for example, a promoter sequence or a restriction endonuclease recognition site.

As used herein, the term “HIV” refers to human immunodeficiency virus. HIV can be classified into two major subtypes (HIV-1 and HIV-2), each of which has many subtypes. In some embodiments, a human subject is infected with the HIV-1 or HIV-2 subtypes.

As used herein, the term “SIV” refers to simian immunodeficiency virus. It is recognized that SIV as a species of retrovirus can cause diseases in monkeys similar to that caused by HIV in humans.

As used herein, the term “viral infection” describes a diseased state in which a virus invades healthy cells, uses the cell's reproductive machinery to multiply or replicate and ultimately lyse the cell, resulting in cell death, release of viral particles, and the infection of other cells by the newly produced progeny viruses. Latent infection by certain viruses, e.g., HIV-1, is also a possible result of viral infection.

As used herein, the term “vaccine” refers to a substance administered to trigger or stimulate an immune response against a particular disease, such as HIV or SIV infection. The term vaccine comprises preventative vaccines and therapeutic vaccines. Preventative vaccines are designed to prevent a subject from acquiring a particular disease, such as HIV or SIV infection, or to only have a mild case of the disease. Therapeutic vaccines are intended to improve immune response to or alleviate symptoms of specific diseases.

The term “vaccine efficacy” as used herein refers to the percentage of reduction in disease incidence in a vaccinated group compared to an unvaccinated group under optimal or controlled conditions such as clinical trials.

The term “vaccine effectiveness” or “effectiveness of the vaccine” as used herein refers to the ability of a vaccine to reduce the risk of or to prevent outcomes of interest under typical field conditions. For example, in certain embodiments, vaccine effectiveness is determined based on the ability of the vaccine to reduce a subject's chances of acquiring a disease after exposure to the disease. A vaccine for a disease, such as HIV or SIV, may be considered effective if, after receiving the vaccine, a subject's chances of acquiring the disease are statistically significantly reduced.

The term “protective immunity” as used herein refers to the immunity of a subject induced by vaccination that offers protection against a subsequent infection of the causative agent of the disease of interest to which the vaccine is developed.

The term “correlate” as used herein refers to an attribute that is statistically associated with an endpoint in a vaccination study.

As used herein, “antibody-dependent cellular phagocytosis (ADCP)” refers to Fc receptor-dependent function of antibody-dependent cellular phagocytosis that provides mechanisms for clearance of virus and virus-infected cells by cells including monocytes and macrophages, as well as for stimulation of downstream adaptive immune responses by facilitating antigen presentation, or by stimulating the secretion of inflammatory mediators.

As used herein, the term “construct” refers to a recombinant nucleotide sequence, such as a recombinant nucleic acid molecule, that has been generated for the purpose of the expression of a specific nucleotide sequence(s), or is to be used in the construction of other recombinant nucleotide sequences.

As used herein, the term “viral vector” refers to either a nucleic acid molecule (e.g., a plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In particular, the terms “lentiviral vector,” “lentiviral expression vector,” etc. may be used to refer to lentiviral particles and/or lentiviral transfer plasmids of certain embodiments as described herein. The phrase “essential lentiviral protein” as used herein refers to those viral protein(s), other than envelope protein, that are required for the lentiviral life cycle. Essential lentiviral proteins may include those required for reverse transcription and integration and for the packaging (e.g., encapsidation) of a retroviral genome.

As used herein, the terms “subject,” “patient,” “individual,” and “host” used interchangeably herein, refer to a mammal, including, but not limited to, murines, felines, simians, humans, mammalian farm animals, mammalian sport animals, and mammalian pets. The term includes mammals that are infected with as well as those that are susceptible to infection by an immunodeficiency virus. In certain embodiments of all aspects of the disclosure, the term refers to a human infected with HIV. In certain embodiments of all aspects of the disclosure, the term refers to a non-human primate infected with SIV.

HUGO Gene Nomenclature Committee (HGNC) annotations are used to describe the genes discussed herein, all of which represent currently known gene sequences. The following Table 1 lists the HGNC annotations, Ensemble gene annotations, Entrezgene numbers, and gene name descriptions for genes discussed herein:

TABLE 1
HGNC and Ensembl Gene Annotations

| HGNC Symbol | HGNC | Ensembl | NCBI | |
|---|---|---|---|---|
| (SEQ ID NO:) | No. | Annotation | No. | Description |
| ACSL1 | 3569 | ENSG00000151726.14 | 2180 | acyl-CoA synthetase |
| (SEQ ID NO: 1) | | | | long-chain family |
| | | | | member 1 |
| ADAM9 | 216 | ENSG00000168615.12 | 8754 | ADAM |
| (SEQ ID NO: 2) | | | | metallopeptidase domain |
| | | | | 9 |
| AHNAK | 347 | ENSG00000124942.14 | 79026 | AHNAK nucleoprotein |
| (SEQ ID NO: 3) | | | | |
| ALDH3B1 | 410 | ENSG00000006534.16 | 221 | aldehyde |
| (SEQ ID NO: 4) | | | | dehydrogenase 3 family |
| | | | | member B1 |
| ALDOA | 414 | ENSG00000149925.20 | 226 | aldolase, fructose- |
| (SEQ ID NO: 5) | | | | bisphosphate A |
| AMPD2 | 469 | ENSG00000116337.19 | 271 | adenosine |
| (SEQ ID NO: 6) | | | | monophosphate |
| | | | | deaminase 2 |
| AOAH | 548 | ENSG00000136250.12 | 313 | acyloxyacyl hydrolase |
| (SEQ ID NO: 7) | | | | |
| AP2S1 | 565 | ENSG00000042753.11 | 1175 | adaptor related protein |
| (SEQ ID NO: 8) | | | | complex 2 subunit sigma |
| | | | | 1 |
| APOBR | 24087 | ENSG00000184730.11 | 55911 | apolipoprotein B |
| (SEQ ID NO: 9) | | | | receptor |
| ARHGEF10L | 25540 | ENSG00000074964.17 | 55160 | Rho guanine nucleotide |
| (SEQ ID NO: 10) | | | | exchange factor 10 like |
| ARRB1 | 711 | ENSG00000137486.17 | 408 | arrestin beta 1 |
| (SEQ ID NO: 11) | | | | |
| ATF3 | 785 | ENSG00000162772.17 | 467 | activating transcription |
| (SEQ ID NO: 12) | | | | factor 3 |
| BHLHE40 | 1046 | ENSG00000134107.5 | 8553 | basic helix-loop-helix |
| (SEQ ID NO: 13) | | | | family member e40 |
| CAMKK2 | 1470 | ENSG00000110931.19 | 10645 | calcium/calmodulin |
| (SEQ ID NO: 14) | | | | dependent protein kinase |
| | | | | 2 |
| CCR1 | 1602 | ENSG00000163823.4 | 1230 | C-C motif chemokine |
| (SEQ ID NO: 15) | | | | receptor 1 |
| CD14 | 1628 | ENSG00000170458.14 | 929 | CD14 molecule |
| (SEQ ID NO: 16) | | | | |
| CD68 | 1693 | ENSG00000129226.14 | 968 | CD68 molecule |
| (SEQ ID NO: 17) | | | | |
| CD86 | 1705 | ENSG00000114013.16 | 942 | CD86 molecule |
| (SEQ ID NO: 18) | | | | |
| CD163 | 1631 | ENSG00000177575.12 | 9332 | CD163 molecule |
| (SEQ ID NO: 19) | | | | |
| CD302 | 30843 | ENSG00000241399.7 | 9936 | CD302 molecule |
| (SEQ ID NO: 20) | | | | |
| CD4 | 1678 | ENSG00000010610.10 | 920 | CD4 molecule |
| (SEQ ID NO: 21) | | | | |
| CDC42EP3 | 16943 | ENSG00000163171.8 | 10602 | CDC42 effector protein |
| (SEQ ID NO: 22) | | | | 3 |
| CDC42EP4 | 17147 | ENSG00000179604.10 | 23580 | CDC42 effector protein |
| (SEQ ID NO: 23) | | | | 4 |
| CEBPA | 1833 | ENSG00000245848.3 | 1050 | CCAAT enhancer |
| (SEQ ID NO: 24) | | | | binding protein alpha |
| CEBPD | 1835 | ENSG00000221869.5 | 1052 | CCAAT enhancer |
| (SEQ ID NO: 25) | | | | binding protein delta |
| CLEC4A | 13257 | ENSG00000111729.14 | 50856 | C-type lectin domain |
| (SEQ ID NO: 26) | | | | family 4 member |
| CPD | 2301 | ENSG00000108582.12 | 1362 | carboxypeptidase D |
| (SEQ ID NO: 27) | | | | |
| CPPED1 | 25632 | ENSG00000103381.12 | 55313 | calcineurin like |
| (SEQ ID NO: 28) | | | | phosphoesterase domain |
| | | | | containing 1 |
| CREB5 | 16844 | ENSG00000146592.17 | 9586 | cAMP responsive |
| (SEQ ID NO: 29) | | | | element binding protein |
| | | | | 5 |
| CREG1 | 2351 | ENSG00000143162.9 | 8804 | cellular repressor of |
| (SEQ ID NO: 30) | | | | E1A stimulated genes 1 |
| CSF1R | 2433 | ENSG00000182578.13 | 1436 | colony stimulating |
| (SEQ ID NO: 31) | | | | factor 1 receptor |
| CSF3R | 2439 | ENSG00000119535.17 | 1441 | colony stimulating |
| (SEQ ID NO: 32) | | | | factor 3 receptor |
| CST1 | 2473 | ENSG00000170373.8 | 1469 | cystatin SN |
| (SEQ ID NO: 33) | | | | |
| CST3 | 2475 | ENSG00000101439.9 | 1471 | cystatin C |
| (SEQ ID NO: 34) | | | | |
| CTBP2 | 2495 | ENSG00000175029.17 | 1488 | C-terminal binding |
| (SEQ ID NO: 35) | | | | protein 2 |
| CTNNA1 | 2509 | ENSG00000044115.21 | 1495 | catenin alpha 1 |
| (SEQ ID NO: 36) | | | | |
| CTSD | 2529 | ENSG00000117984.14 | 1509 | cathepsin D |
| (SEQ ID NO: 37) | | | | |
| DAPK1 | 2674 | ENSG00000196730.13 | 1612 | death-associated protein |
| (SEQ ID NO: 38) | | | | kinase 1 |
| DOCK5 | 23476 | ENSG00000147459.18 | 80005 | dedicator of cytokinesis |
| (SEQ ID NO: 39) | | | | 5 |
| DUSP6 | 3072 | ENSG00000139318.8 | 1848 | dual specificity |
| (SEQ ID NO: 40) | | | | phosphatase 6 |
| EFHD2 | 28670 | ENSG00000142634.13 | 79180 | EF-hand domain family |
| (SEQ ID NO: 41) | | | | member D2 |
| EMILIN2 | 19881 | ENSG00000132205.11 | 84034 | elastin microfibril |
| (SEQ ID NO: 42) | | | | interface 2 |
| ENO1 | 3350 | ENSG00000074800.16 | 2023 | enolase 1 |
| (SEQ ID NO: 43) | | | | |
| ERMAP | 15743 | ENSG00000164010.15 | 114625 | erythroblast membrane- |
| (SEQ ID NO: 44) | | | | associated protein |
| OTULINL | 25629 | ENSG00000145569.6 | 54491 | OUT deubiquitinase |
| (FAM105A) | | | | with linear linkage |
| (SEQ ID NO: 45) | | | | specificity like |
| FBXL5 | 13602 | ENSG00000118564.15 | 26234 | F-box and leucine rich |
| (SEQ ID NO: 46) | | | | repeat protein 5 |
| FCGRT | 3621 | ENSG00000104870.13 | 2217 | Fc fragment of IgG |
| (SEQ ID NO: 47) | | | | receptor and transporter |
| FLVCR2 | 20105 | ENSG00000119686.1 | 55640 | FLVCR2 heme |
| (SEQ ID NO: 48) | | | | transporter 2 |
| FZD1 | 4038 | ENSG00000157240.4 | 8321 | frizzled class receptor 1 |
| (SEQ ID NO: 49) | | | | |
| GAA | 4065 | ENSG00000171298.13 | 2548 | glucosidase alpha, acid |
| (SEQ ID NO: 50) | | | | |
| GABARAPL1 | 4068 | ENSG00000139112.11 | 23710 | GABA type A receptor |
| (SEQ ID NO: 51) | | | | associated protein like 1 |
| GAS7 | 4169 | ENSG00000007237.18 | 8522 | growth arrest-specific 7 |
| (SEQ ID NO: 52) | | | | |
| GLA | 4296 | ENSG00000102393.11 | 2717 | galactosidase alpha |
| (SEQ ID NO: 53) | | | | |
| GNAO1 | 4389 | ENSG00000087258.15 | 2775 | G protein subunit alpha |
| (SEQ ID NO: 54) | | | | ol |
| GNS | 4422 | ENSG00000135677.11 | 2799 | glucosamine (N-acetyl)- |
| (SEQ ID NO: 55) | | | | 6-sulfatase |
| GRN | 4601 | ENSG00000030582.18 | 2896 | granulin precursor |
| (SEQ ID NO: 56) | | | | |
| H2AFY | 4740 | ENSG00000113648.16 | 9555 | macroH2A.1 histone |
| (MACROH2A1) | | | | (previously H2A histone |
| (SEQ ID NO: 57) | | | | family member Y) |
| HBEGF | 3059 | ENSG00000113070.8 | 1839 | heparin binding EGF |
| (SEQ ID NO: 58) | | | | like growth factor |
| HEXB | 4879 | ENSG00000049860.14 | 3074 | hexosaminidase subunit |
| (SEQ ID NO: 59) | | | | beta |
| HMOX1 | 5013 | ENSG00000100292.17 | 3162 | heme oxygenase 1 |
| (SEQ ID NO: 60) | | | | |
| HPSE | 5164 | ENSG00000173083.15 | 10855 | heparanase |
| (SEQ ID NO: 61) | | | | |
| HSBP1 | 5203 | ENSG00000230989.7 | 3281 | heat shock factor |
| (SEQ ID NO: 62) | | | | binding protein 1 |
| ICAM1 | 5344 | ENSG00000090339.9 | 3383 | intercellular adhesion |
| (SEQ ID NO: 63) | | | | molecule 1 |
| ID2 | 5361 | ENSG00000115738.10 | 3398 | inhibitor of DNA |
| (SEQ ID NO: 64) | | | | binding 2 |
| IL17RA | 5985 | ENSG00000177663.14 | 23765 | interleukin 17 receptor |
| (SEQ ID NO: 65) | | | | A |
| IMPA2 | 6051 | ENSG00000141401.12 | 3613 | inositol |
| (SEQ ID NO: 66) | | | | monophosphatase 2 |
| IRAK3 | 17020 | ENSG00000090376.11 | 11213 | interleukin-1 receptor- |
| (SEQ ID NO: 67) | | | | associated kinase 3 |
| KIAA0513 | 29058 | ENSG00000135709.12 | 9764 | KIAA0513 |
| (SEQ ID NO: 68) | | | | |
| KLF10 | 11810 | ENSG00000155090.15 | 7071 | Kruppel like factor 10 |
| (SEQ ID NO: 69) | | | | |
| LAMP2 | 6501 | ENSG00000005893.16 | 3920 | lysomal-associated |
| (SEQ ID NO: 70) | | | | membrane protein 2 |
| LEPROT | 29477 | ENSG00000213625.9 | 54741 | leptin receptor |
| (SEQ ID NO: 71) | | | | overlapping transcript |
| LGALS2 | 6562 | ENSG00000100079.7 | 3957 | galectin 2 |
| (SEQ ID NO: 72) | | | | |
| LMNA | 6636 | ENSG00000160789.20 | 4000 | lamin A/C |
| (SEQ ID NO: 73) | | | | |
| LRPAP1 | 6701 | ENSG00000163956.13 | 4043 | LDL receptor related |
| (SEQ ID NO: 74) | | | | protein associated |
| | | | | protein 1 |
| LST1 | 14189 | ENSG00000204482.10 | 7940 | leukocyte specific |
| (SEQ ID NO: 75) | | | | transcript 1 |
| MAFB | 6408 | ENSG00000204103.4 | 9935 | MAF bZIP transcription |
| (SEQ ID NO: 76) | | | | factor B |
| MAPKAPK3 | 6888 | ENSG00000114738.11 | 7867 | mitogen-activated |
| (SEQ ID NO: 77) | | | | protein kinase-activated |
| | | | | protein kinase 3 |
| MCL1 | 6943 | ENSG00000143384.13 | 4170 | MCL1-BCL2 family |
| (SEQ ID NO: 78) | | | | apoptosis regulator |
| MICAL2 | 24693 | ENSG00000133816.15 | 9645 | microtubule associated |
| (SEQ ID NO: 79) | | | | monooxygenase, |
| | | | | calponin and LIM |
| | | | | domain containing 2 |
| MGST2 | 7063 | ENSG00000085871.9 | 4258 | microsomal glutathione |
| (SEQ ID NO: 80) | | | | S-transferase 2 |
| MPP1 | 7219 | ENSG00000130830.15 | 4354 | membrane palmitoylated |
| (SEQ ID NO: 81) | | | | protein 1 |
| MYO1F | 7600 | ENSG00000142347.19 | 4542 | myosin IF |
| (SEQ ID NO: 82) | | | | |
| NAGA | 7631 | ENSG00000198951.12 | 4668 | alpha-N- |
| (SEQ ID NO: 83) | | | | acetylgalactosaminidase |
| NAMPT | 30092 | ENSG00000105835.12 | 10135 | nicotinamide |
| (SEQ ID NO: 84) | | | | phosphoribosyltransferase |
| NFIL3 | 7787 | ENSG00000165030.4 | 4783 | nuclear factor, |
| (SEQ ID NO: 85) | | | | interleukin 3 regulated |
| NLRP3 | 16400 | ENSG00000162711.17 | 114548 | NLR family, pyrin |
| (SEQ ID NO: 86) | | | | domain containing 3 |
| NPL | 16781 | ENSG00000135838.13 | 80896 | N-acetylneuraminate |
| (SEQ ID NO: 87) | | | | pyruvate lyase |
| OGFRL1 | 21378 | ENSG00000119900.9 | 79627 | opioid growth factor |
| (SEQ ID NO: 88) | | | | response like 1 |
| PHACTR2 | 20956 | ENSG00000112419.14 | 9749 | phosphatase and actin |
| (SEQ ID NO: 89) | | | | regulator 2 |
| PGD | 8891 | ENSG00000142657.21 | 5226 | phosphogluconate |
| (SEQ ID NO: 90) | | | | dehydrogenase |
| PKM2 | 9021 | ENSG00000067225.18 | 5315 | pyruvate kinase M1/2 |
| (SEQ ID NO: 91) | | | | |
| PSAP | 9498 | ENSG00000197746.14 | 5660 | prosaposin |
| (SEQ ID NO: 92) | | | | |
| PSTPIP1 | 9580 | ENSG00000140368.13 | 9051 | proline-serine-threonine |
| (SEQ ID NO: 93) | | | | phosphatase interacting |
| | | | | protein 1 |
| PTGER2 | 9594 | ENSG00000125384.7 | 5732 | prostaglandin E receptor |
| (SEQ ID NO: 94) | | | | 2 |
| PXN | 9718 | ENSG00000089159.16 | 5829 | paxillin |
| (SEQ ID NO: 95) | | | | |
| PYGL | 9725 | ENSG00000100504.17 | 5836 | glycogen phosphorylase |
| (SEQ ID NO: 96) | | | | L |
| QPCT | 9753 | ENSG00000115828.17 | 25797 | glutaminyl-peptide |
| (SEQ ID NO: 97) | | | | cyclotransferase |
| RAB20 | 18260 | ENSG00000139832.5 | 55647 | RAB20, member RAS |
| (SEQ ID NO: 98) | | | | oncogene family |
| RAB27A | 9766 | ENSG00000069974.16 | 5873 | RAB27A, member RAS |
| (SEQ ID NO: 99) | | | | oncogene family |
| RAB32 | 9772 | ENSG00000118508.5 | 10981 | RAB32, member RAS |
| (SEQ ID NO: 100) | | | | oncogene family |
| RBM47 | 30358 | ENSG00000163694.15 | 54502 | RNA binding motif |
| (SEQ ID NO: 101) | | | | protein 47 |
| REEP4 | 26176 | ENSG00000168476.12 | 80346 | receptor accessory |
| (SEQ ID NO: 102) | | | | protein 4 |
| RGS10 | 9992 | ENSG00000148908.15 | 6001 | regulator of G protein |
| (SEQ ID NO: 103) | | | | signaling 10 |
| RNF130 | 18280 | ENSG00000113269.14 | 55819 | ring finger protein 130 |
| (SEQ ID NO: 104) | | | | |
| RRAGD | 19903 | ENSG00000025039.15 | 58528 | ras-related GTP binding |
| (SEQ ID NO: 105) | | | | D |
| RTN1 | 10467 | ENSG00000139970.17 | 6252 | reticulon 1 |
| (SEQ ID NO: 106) | | | | |
| RXRA | 10477 | ENSG00000186350.12 | 6256 | retinoid X receptor alpha |
| (SEQ ID NO: 107) | | | | |
| SCARB2 | 1665 | ENSG00000138760.10 | 950 | scavenger receptor class |
| (SEQ ID NO: 108) | | | | B member 2 |
| SDCBP | 10662 | ENSG00000137575.12 | 6386 | syndecan binding |
| (SEQ ID NO: 109) | | | | protein (syntenin) |
| SEMA4A | 10729 | ENSG00000196189.13 | 64218 | semaphorin 4A |
| (SEQ ID NO: 110) | | | | |
| SERINC5 | 18825 | ENSG00000164300.17 | 256987 | serine incorporator 5 |
| (SEQ ID NO: 111) | | | | |
| SIRPA | 9662 | ENSG00000198053.11 | 140885 | signal-regulatory protein |
| (SEQ ID NO: 112) | | | | alpha |
| SIRPB1 | 15928 | ENSG00000101307.15 | 10326 | signal regulatory protein |
| (SEQ ID NO: 113) | | | | beta 1 |
| SLC27A3 | 10997 | ENSG00000143554.14 | 11000 | solute carrier family 27 |
| (SEQ ID NO: 114) | | | | member 3 |
| SLC31A2 | 11017 | ENSG00000136867.11 | 1318 | solute carrier family 31 |
| (SEQ ID NO: 115) | | | | member 2 |
| SLC36A1 | 18761 | ENSG00000123643.13 | 206358 | solute carrier family 36 |
| (SEQ ID NO: 116) | | | | member 1 |
| SLCO3A1 | 10952 | ENSG00000176463.14 | 28232 | solute carrier organic |
| (SEQ ID NO: 117) | | | | anion transporter family |
| | | | | member 3A1 |
| SORT1 | 11186 | ENSG00000134243.12 | 6272 | sortilin 1 |
| (SEQ ID NO: 118) | | | | |
| ST3GAL6 | 18080 | ENSG00000064225.12 | 10402 | ST3 beta-galactoside |
| (SEQ ID NO: 119) | | | | alpha-2,3- |
| | | | | sialyltransferase 6 |
| STX11 | 11429 | ENSG00000135604.10 | 8676 | syntaxin 11 |
| (SEQ ID NO: 120) | | | | |
| SULT1A2 | 11454 | ENSG00000197165.11 | 6799 | sulfotransferase family |
| (SEQ ID NO: 121) | | | | 1A member 2 |
| TBC1D8 | 17791 | ENSG00000204634.12 | 11138 | TBC1 domain family |
| (SEQ ID NO: 122) | | | | member 8 |
| TBXAS1 | 11609 | ENSG00000059377.17 | 6916 | thromboxane A synthase |
| (SEQ ID NO: 123) | | | | 1 |
| TIMP2 | 11821 | ENSG00000035862.12 | 7077 | TIMP metallopeptidase |
| (SEQ ID NO: 124) | | | | inhibitor 2 |
| TKT | 11834 | ENSG00000163931.16 | 7086 | transketolase |
| (SEQ ID NO: 125) | | | | |
| TNFRSF1B | 11917 | ENSG00000028137.19 | 7133 | tumor necrosis factor |
| (SEQ ID NO: 126) | | | | receptor superfamily |
| | | | | member 1B |
| TNFSF12 | 11927 | ENSG00000239697.11 | 8742 | tumor necrosis factor |
| (SEQ ID NO: 127) | | | | superfamily member 12 |
| TNFSF13 | 11928 | ENSG00000161955.16 | 8741 | tumor necrosis factor |
| (SEQ ID NO: 128) | | | | superfamily member 13 |
| TRIB1 | 16891 | ENSG00000173334.4 | 10221 | tribbles pseudokinase 1 |
| (SEQ ID NO: 129) | | | | |
| TYROBP | 12449 | ENSG00000011600. | 730512 | TYRO protein tyrosine |
| (SEQ ID NO: 130) | | | | kinase binding protein |
| VIM | 12692 | ENSG00000026025.16 | 7431 | vimentin |
| (SEQ ID NO: 131) | | | | |
| VEGFA | 12680 | ENSG00000112715. | 7422 | vascular endothelial |
| (SEQ ID NO: 132) | 23 | | | growth factor A |
| VPS37C | 26097 | ENSG00000167987.11 | 55048 | VPS37C, ESCRT-I |
| (SEQ ID NO: 133) | | | | subunit |
| WDFY3 | 20751 | ENSG00000163625.15 | 23001 | WD repeat and FYVE |
| (SEQ ID NO: 134) | | | | domain containing 3 |
| ZDHHC7 | 18459 | ENSG00000153786.12 | 55625 | zinc finger DHHC-type |
| (SEQ ID NO: 135) | | | | palmitoyltransferase 7 |
| ZNF467 | 23154 | ENSG00000181444.13 | 168544 | zinc finger protein 467 |
| (SEQ ID NO: 136) | | | | |

Disclosed herein is an orthogonal approach to identifying differentially expressed genes or gene signatures that correlate to immunity against a virus and using those differentially expressed genes or gene signatures to evaluate vaccine efficacy or the ability of a vaccine or vaccine candidate to confer protective immunity to a subject. The gene signatures disclosed herein may be a broad indicator of effective vaccination against a range of RNA viruses, including, for example, HIV, SIV, influenza, and yellow fever vaccines.

Vaccines

Disclosed herein are methods for evaluating vaccine efficacy and vaccine effectiveness, as well as methods for predicting protective immunity, in subjects who have received vaccination against HIV or SIV infection. The vaccine may comprise a primer vaccine and a booster vaccine. In some embodiments of all aspects of the disclosure, an HIV vaccine is used. The HIV vaccine may comprise one or more primer vaccines and one or more booster vaccines. The HIV vaccine may comprise one or more recombinant proteins, one or more recombinant nucleic acid molecules, and combination thereof. In certain embodiments of all aspects of the disclosure, the one or more recombinant nucleic acid vaccine molecules comprise recombinant viral vector vaccines. The recombinant viral vectors may include, but are not limited to, vaccinia vectors, adenovirus vectors, adeno-associated virus vectors, canarypox virus vectors, herpes simplex virus vectors, human papillomavirus vectors, modified vaccinia Ankara (MVA) vectors, and retroviral vectors.

In some embodiments of all aspects of the disclosure, the recombinant viral vaccine is ALVAC-HIV (vCP1521). ALVAC-HIV (vCP1521) is a recombinant canarypox vaccine developed by Virogenetics Corporation (Troy, N.Y.) and manufactured by Sanofi Pasteur (Marcy-l'Etoille, France). The recombinant canarypox was genetically engineered to express HIV-1 Gag and Pro (subtype B LAI strain) and CRFO1_AE (subtype E) HIV-1 gp120 (92TH023) linked to the transmembrane 3 anchoring portion of gp41 (LAI). In some embodiments of all aspects of the disclosure, the recombinant viral vaccine is AIDSVAX® B/E. AIDSVAX® B/E (Global Solutions for Infectious Diseases, South San Francisco, Calif.) is a bivalent HIV gp120 envelope glycoprotein vaccine containing a subtype E envelope from the HIV-1 strain A244 (CM244) and a subtype B envelope from the HIV-1 MN produced in Chinese hamster ovary cell lines. In some embodiments of all aspects of the disclosure, the recombinant viral vaccine is Ad26.Mos.HIV may be used. Ad26.Mos.HIV is an HIV vaccine expressing mosaic HIV-1 envelope (Env)/Gag/Pol antigens. In some embodiments of all aspects of the disclosure, a booster vaccine may be used including, but not limited to, Ad26.Mos.HIV or modified vaccinia Ankara (MVA) vectors with or without high-dose (250 μg) or low-dose (50 μg) aluminum adjuvanted clade C Env gp140 protein as described, for example, by Barouch II.

As used herein, the terms “HIV antigen,” “antigenic polypeptide of an HIV,” “HIV antigenic polypeptide,” “HIV antigenic protein,” “HIV immunogenic polypeptide,” and “HIV immunogen” all refer to a polypeptide capable of inducing an immune response, e.g., a humoral and/or cellular mediated immune response, against HIV in a subject. The HIV antigen can be a protein of HIV (e.g., HIV gag, pol and env gene products), a fragment or epitope thereof, or a combination of multiple HIV proteins or portions thereof, that can induce an immune response against HIV in a subject. An HIV antigen is capable of protective immunity in a subject.

In certain embodiments of all aspects of the disclosure, the HIV antigen can be an HIV-1 or HIV-2 antigen or fragment(s) thereof. Examples of HIV antigens include, but are not limited to gag, pol, and env gene products, which encode structural proteins and essential enzymes. Gag, pol, and env gene products are synthesized as polyproteins, which are further processed into multiple other protein products. The primary protein product of the gag gene is the viral structural protein gag polyprotein, which is further processed into MA, CA, SP1, NC, SP2, and P6 protein products. The pol gene encodes viral enzymes (Pol, polymerase), and the primary protein product is further processed into RT, RNase H, IN, and PR protein products. The env gene encodes structural proteins, specifically glycoproteins of the virion envelope. The primary protein product of the env gene is gp160, which is further processed into gp120 and gp41. A heterologous nucleic acid sequence as disclosed herein may encode a gag, env, and/or pol gene product, or portion thereof.

In some embodiments of all aspects of the disclosure, an SIV vaccine is used. An SIV vaccine may comprise Ad26 vectors expressing SIVsmE543 Env, Gag, and Pol and boosted with AS01B-adjuvanted SIVmac32H Env gp140, and Ad35 vectors expressing SIVsmE543 Env/Gag/Pol antigens as described, for example, by Barouch I. In some embodiments, the SIV vaccine can be Ad26, Ad35, or Ad5HVR48 vectors expressing Env/Gag/Pol and/or 0.25 mg purified Env gp140 protein with AS01B adjuvant system as described, for example, by Barouch I.

Evaluating Vaccine Efficacy and Effectiveness and Predicting Protective Immunity

Disclosed herein are methods of evaluating vaccine efficacy and/or effectiveness against a virus, such as HIV or SIV, in a subject, for example in a subject that has been vaccinated against the virus. In certain embodiments, the method of evaluating vaccine efficacy and/or effectiveness comprises detecting expression of multiple genes and/or gene products in a biological sample from the subject, wherein the multiple genes and/or gene products have been identified as a differentially expressed gene set associated with efficacy of a vaccine; calculating a first composite gene expression score (GES) for the differentially expressed gene set in the biological sample from the subject; and calculating an average second composite GES for the differentially expressed gene set in a plurality of biological samples from subjects that have been vaccinated with the candidate HIV and SIV vaccine and are not immune to HIV or SIV. In certain embodiments, if the first composite GES is greater than the average second composite GES, it indicates effectiveness of the vaccine. In certain embodiments, the vaccine may be a candidate vaccine whose efficacy is being evaluated in a study population, for example a non-human primate study population or a human population. In certain embodiments, the method further comprises a step of identifying the differentially expressed gene set associated with efficacy and/or effectiveness of the vaccine. In certain embodiments, the method further comprises a step of adjusting treatment options for the subject based on the predicted effectiveness of the vaccine in the subject.

Disclosed herein are methods of predicting protective immunity against a virus, such as HIV or SIV, in a subject, for example in a subject that has been vaccinated against the virus. In certain embodiments, the method of predicting protective immunity comprises detecting expression of multiple genes and/or gene products in a biological sample from the subject, wherein the multiple genes and/or gene products have been identified as a differentially expressed gene set associated with protective immunity against HIV or SIV; calculating a first composite gene expression score (GES) for the differentially expressed gene set in the biological sample from the subject; and calculating an average second composite GES for the differentially expressed gene set in a plurality of biological samples from subjects that have been vaccinated with the candidate HIV and SIV vaccine and are not immune to HIV or SIV. In certain embodiments, if the first composite GES is greater than the average second composite GES, it indicates that the subject that has been vaccinated with the vaccine has protective immunity against the virus. In certain embodiments, the vaccine may be a candidate vaccine whose efficacy is being evaluated in a study population, for example a non-human primate study population or a human population. In other embodiments, a biological sample from the subject is being evaluated to determine whether protective immunity has been conferred from a particular vaccine or candidate vaccine to the subject. In certain embodiments, the method further comprises a step of identifying the differentially expressed gene set associated with protective immunity against HIV or SIV. In certain embodiments, the method further comprises a step of adjusting treatment options for the subject based on whether the subject is indicated to have protective immunity against HIV or SIV.

Also disclosed herein are methods of collecting data for evaluating protective immunity against a virus, such as HIV or SIV, in a subject, for example a subject that has been vaccinated against the virus. In certain embodiments, the method of collecting data for evaluating protective immunity comprises detecting expression of multiple genes and/or gene products in a biological sample from the subject, wherein the multiple genes and/or gene products have been identified as a differentially expressed gene set associated with protective immunity against HIV or SIV, calculating a first composite GES based on the expression of the genes, and calculating an average second composite GES based on expression of the genes in the differentially expressed gene set in a plurality of biological samples from subjects that are not immune to the virus. In certain embodiments, the subjects that are not immune to the virus have been vaccinated against the virus but did not acquire protective immunity; in some embodiments, the subjects that are not immune to the virus have not been vaccinated against the virus and have acquired the virus. In certain embodiments, the method further comprises a step of identifying the differentially expressed gene set associated with protective immunity against HIV or SIV. In certain embodiments, the method further comprises a step of adjusting treatment options for the subject based on whether the subject is identified as having protective immunity against HIV or SIV based on the GES scores.

A GES may be calculated for a gene or gene set based on the differential expression of the genes and/or gene products relative to a normalized expression for the genes and/or gene products. The GES may be calculated by calculating the Z score or an average of the Z scores of a gene or gene product or gene set in a sample or collection of samples relative to the mean differential gene expression for the particular gene or gene product or gene set. Normalized gene expression may, in certain embodiments, be measured by calculating an average gene or gene product expression from a biological sample or pool of biological samples from subjects that have not been vaccinated with the vaccine and are not infected with the virus. In certain embodiments of all aspects of the disclosure, normalized gene expression for a gene or gene product may be available, for example in a published online database.

In certain embodiments of all aspects of the disclosure, the Z score is calculated as (X−μ)/std(X), wherein X is the gene expression score for a particular gene and/or gene product in a sample, μ is the mean expression score of the genes and/or gene products in the gene set for that sample, and std(X) is the standard deviation for the mean expression score. In certain embodiments of all aspects of the disclosure, std(X) may calculated as the square root of the variance (σ), wherein σ=[(X−m)²]/(n−1), wherein n equals the number of samples in the sample set. In certain embodiments of all aspects of the disclosure, the GES is correlated to protection from viral acquisition in that the higher the GES, the greater the protection from viral acquisition. The composite GES is the average GES calculated for a gene or gene set. A GES may be calculated based on measuring or detecting mRNA, cDNA, or protein levels of genes in the differentially expressed gene set.

In certain embodiments of all aspects of the disclosure, the higher the first composite GES relative to the second composite GES score, the less likely a subject is to acquire a particular virus if the subject is exposed to the virus, i.e., the greater the likelihood that the vaccine has been effective to confer immunity to the virus.

In certain embodiments of all aspects of the disclosure, the vaccine is a candidate vaccine, such as a candidate HIV or SIV vaccine. In certain embodiments, the vaccine comprises an Ad26 vector, an envelope glycoprotein, such as an HIV or SIV envelope glycoprotein, or a combination of both an Ad26 vector and an envelope glycoprotein.

A differentially expressed gene set associated with protective immunity may be identified by any means known in the art. In certain embodiments of all aspects of the disclosure, the differentially expressed gene set may have been previously identified. In other embodiments of all aspects of the disclosure, the differentially expressed gene set may be identified using any of the methods disclosed herein for the detection of gene expression. According to various embodiments disclosed herein, the differentially expressed gene set is associated with a B-cell gene signature. According to various embodiments disclosed herein, the differentially expressed gene set is associated with a monocyte gene signature.

Differentially Expressed Gene Set Indicative of Vaccine Efficacy and Protective Immunity

Disclosed herein is a differentially expressed gene set that can be used to calculate composite GES in the methods disclosed herein. In certain embodiments of all aspects of the disclosure, the differentially expressed gene set disclosed herein may be used to evaluate protective immunity against an RNA virus, such as HIV or SIV. In certain embodiments of all aspects of the disclosure, the differentially expressed gene set can be used to evaluate protective immunity in a human subject or group of human subjects. In other embodiments of all aspects of the disclosure, the differentially expressed gene set can be used to evaluate protective immunity in a non-human primate subject or group of non-human primate subjects.

As discussed above, two independent preclinical non-human primate (NHP) challenge studies, Barouch I and Barouch II, showed partial vaccine efficacy with an Ad26-based HIV-1 vaccine candidate that elicited strong antibody responses. In Barouch I, the protective efficacy of an Ad26 vector priming followed by boosting with a purified envelope (Env) glycoprotein was evaluated. The priming Ad26 vector expressed SIVsmE543 Eng/Gag/Pol antigens, and the booster comprised AS01B-adjuvanted SIVmac32H Env gp140. It was found that 50% of the vaccinated NHPs exhibited protection after multiple intrarectal challenges with SIVmac251, while 100% of the non-vaccinated NHP controls exhibited viral acquisition. In Barouch II, a vaccine regimen comprising a mosaic Ad26/Ad26 vaccine followed by a gp140 vaccine boost was evaluated after multiple intrarectal challenges with SHIV-SF162P3.

Transcriptomic profiling of cryopreserved samples from these studies by RNA sequencing at time points after vaccination but prior to viral challenge identified a differentially expressed gene set in B cells that associated with protection from viral acquisition in both studies. Protection was also observed in a human trial that previously showed vaccine efficacy and in two additional NHP studies, where canarypox-based vaccine regimens were evaluated. See Rerks-Ngarm, S. et al., Vaccination with ALVAC and AIDSVAX to prevent HIV-1 infection in Thailand, NEW ENG. J. MED. 361, 2209-20 (2009); Vaccari, M., et al., Adjuvant-dependent innate and adaptive immune signatures of risk of SIVmac251 acquisition, NAT. MED., 22, 762-70 (2016); and Vaccari, M. et al., HIV vaccine candidate activation of hypoxia and the inflammasome in CD14(+) monoctyes is associated with a decreased risk of SIVmac251 acquisition, NAT. MED. 24, 847-56 (2018). The B-cell gene signature disclosed herein may therefore be associated with a higher magnitude of functional antibody responses in various vaccine regimens, including Ad26-based vaccine and envelope glycoprotein-based vaccine regimens.

In various embodiments, disclosed herein is a method for predicting effectiveness of a vaccine against HIV or SIV in a subject that has been vaccinated with the vaccine, the method comprising detecting expression of multiple genes and/or gene products in a biological sample from the subject, wherein the multiple genes and/or gene products have been identified as a differentially expressed gene set associated with efficacy of the vaccine and comprise at least 3, at least 10, at least 15, at least 20, at least 25, at least 30, or 32 of the following genes: ACSL1, BHLHE40, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, OGFRL1, PHACTR2, RAB32, REEP4, RNF130, RRAGD, SDCBP, SIRPA, ST3GAL6, TKT, TNFRSF1B, TNFSF13, and VEGFA. The method further comprises calculating a first composite GES for the differentially expressed gene set in the biological sample from the subject, and calculating an average second composition GES for the differentially expressed gene set in a plurality of biological samples from subjects that have been vaccinated with the vaccine and are not immune to the virus, wherein a first composite GES that is greater than the average second composite GES indicates effectiveness of the vaccine. In certain embodiments, the differentially expressed gene set comprises at least BHLHE40, OGFRL1, and TNFSF13. In certain embodiments, the method further comprises identifying the differentially expressed gene set. In certain embodiments, the method further comprises a step of adjusting treatment options for the subject based on the predicted effectiveness of the vaccine in the subject.

In various embodiments, disclosed herein is a method for predicting protective immunity against a virus in a subject that has been vaccinated with a vaccine, the method comprising detecting expression of multiple genes and/or gene products in a biological sample from the subject, wherein the multiple genes and/or gene products have been identified as a differentially expressed gene set associated with protective immunity against HIV or SIV and comprise at least 3, at least 10, at least 15, at least 20, at least 25, at least 30, or 32 of the following genes: ACSL1, BHLHE40, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, OGFRL1, PHACTR2, RAB32, REEP4, RNF130, RRAGD, SDCBP, SIRPA, ST3GAL6, TKT, TNFRSF1B, TNFSF13, and VEGFA. The method further comprises calculating a first composite GES for the differentially expressed gene set in the biological sample from the subject, and calculating an average second composition GES for the differentially expressed gene set in a plurality of biological samples from subjects that have been vaccinated with the vaccine and are not immune to the virus, wherein a first composite GES that is greater than the average second composite GES indicates the subject that has been vaccinated with the candidate vaccine has protective immunity against the virus. In certain embodiments, the differentially expressed gene set comprises at least BHLHE40, OGFRL1, and TNFSF13. In certain embodiments, the method further comprises identifying the differentially expressed gene set. In certain embodiments, the method further comprises a step of adjusting treatment options for the subject based on whether the subject is indicated to have protective immunity against HIV or SIV.

In various embodiments, disclosed herein is a method for predicting effectiveness of a vaccine against HIV or SIV in a subject that has been vaccinated with the vaccine, the method comprising detecting expression levels of multiple genes and/or gene products in a biological sample from the subject, wherein the multiple genes and/or gene products have been identified as a differentially expressed gene set associated with efficacy of the vaccine and comprise at least 5, at least 6, at least 10, at least 20, at least 30, at least 40, at least 50, at least 60, or 62 of the following genes: ACSL1, AHNAK, AOAH, ARHGEF10L, ARRB1, BHLHE40, CAMKK2, CD86, CD163, CDC42EP3, CEBPA, CEBPD, CLEC4A, CPD, CPPED1, CREB5, CREG1, CSF3R, CST1, CTNNA1, DAPK1, DOCK5, EMILIN2, ENO1, ERMAP, FAM105A, FBXL5, GLA, GNS, HBEGF, HEXB, HPSE, HSBPJ, ID2, IRAK3, KIAA0513, LAMP2, LMNA, MAFB, MAPKAPK3, MICAL2, MYOIF, NFIL3, OGFRL1, PGD, PSAP, RAB27A, RNF130, RRAGD, RXRA, SCARB2, SDCBP, SEMA4A, SLC31A2, SLC36A1, STX11, TBC1D8, TNFRSF1B, TNFSF13, VEGFA, VPS37C, and WDFY3. The method further comprises calculating a first composite GES for the differentially expressed gene set in the biological sample from the subject, and calculating an average second composition GES for the differentially expressed gene set in a plurality of biological samples from subjects that have been vaccinated with the vaccine and are not immune to the virus, wherein a first composite GES that is greater than the average second composite GES indicates effectiveness of the vaccine. In certain embodiments, the differentially expressed gene set comprises at least CREB5, DOCK5, ERMAP, IRAK3, OGFRL1, and RNF130. In certain embodiments, the method further comprises identifying the differentially expressed gene set. In certain embodiments, the method further comprises a step of adjusting treatment options for the subject based on the predicted effectiveness of the vaccine in the subject.

In various embodiments, disclosed herein is a method for predicting protective immunity against a virus in a subject that has been vaccinated with a vaccine, the method comprising detecting expression levels of multiple genes and/or gene products in a biological sample from the subject, wherein the multiple genes and/or gene products have been identified as a differentially expressed gene set associated with protective immunity against HIV or SIV and comprise at least 5, at least 6, at least 10, at least 20, at least 30, at least 40, at least 50, at least 60, or 62 of the following genes: ACSL1, AHNAK, AOAH, ARHGEF10L, ARRB1, BHLHE40, CAMKK2, CD86, CD163, CDC42EP3, CEBPA, CEBPD, CLEC4A, CPD, CPPED1, CREB5, CREG1, CSF3R, CST1, CTNNA1, DAPK1, DOCK5, EMILIN2, ENO1, ERMAP, FAM105A, FBXL5, GLA, GNS, HBEGF, HEXB, HPSE, HSBP1, ID2, IRAK3, KIAA0513, LAMP2, LMNA, MAFB, MAPKAPK3, MICAL2, MYOIF, NFIL3, OGFRL1, PGD, PSAP, RAB27A, RNF130, RRAGD, RXRA, SCARB2, SDCBP, SEMA4A, SLC31A2, SLC36A1, STX11, TBC1D8, TNFRSF1B, TNFSF13, VEGFA, VPS37C, and WDFY3. The method further comprises calculating a first composite GES for the differentially expressed gene set in the biological sample from the subject, and calculating an average second composition GES for the differentially expressed gene set in a plurality of biological samples from subjects that have been vaccinated with the vaccine and are not immune to the virus, wherein a first composite GES that is greater than the average second composite GES indicates the subject that has been vaccinated with the candidate vaccine has protective immunity against the virus. In certain embodiments, the differentially expressed gene set comprises at least CREB5, DOCK5, ERMAP, IRAK3, OGFRL1, and RNF130. In certain embodiments, the method further comprises identifying the differentially expressed gene set. In certain embodiments, the method further comprises a step of adjusting treatment options for the subject based on whether the subject is indicated to have protective immunity against HIV or SIV.

In various embodiments, disclosed herein is a method for predicting effectiveness of a vaccine against HIV or SIV in a subject that has been vaccinated with the vaccine, the method comprising detecting expression levels of multiple genes and/or gene products in a biological sample from the subject, wherein the multiple genes and/or gene products have been identified as a differentially expressed gene set associated with efficacy of the vaccine and comprise at least 5, at least 6, at least 10, at least 20, at least 30, at least 40, at least 50, or 52 of the following genes: ALDOA, AOAH, AP2S1, APOBR, ARHGEF10L, ATF3, CD163, CEBPD, CLEC4A, CREB5, CSFIR, CSF3R, CST3, CTNNAL, DOCK5, DUSP6, EFHD2, ERMAP, FCGRT, GAA, GABARAPL1, GNAO, GRN, H2AFY, HBEGF, HSBP1, ICAM1, IRAK3, IL17RA, KIAA0513, KLF10, LST1, MAFB, MCL1, NAGA, NAMPT, NLRP3, OGFRL1, PSAP, PSTPIP1, RAB32, RGS10, RNF130, SIRPA, SLCO3A1, TBC1D8, TKT, TNFSF13, TRIB1, TYROBP, VIM, and WDFY3. The method further comprises calculating a first composite GES for the differentially expressed gene set in the biological sample from the subject, and calculating an average second composition GES for the differentially expressed gene set in a plurality of biological samples from subjects that have been vaccinated with the vaccine and are not immune to the virus, wherein a first composite GES that is greater than the average second composite GES indicates the subject that has been vaccinated with the candidate vaccine has protective immunity against the virus. In certain embodiments, the differentially expressed gene set comprises at least CREB5, DOCK5, ERMAP, IRAK3, OGFRL1, and RNF130. In certain embodiments, the method further comprises identifying the differentially expressed gene set. In certain embodiments, the method further comprises a step of adjusting treatment options for the subject based on the predicted effectiveness of the vaccine in the subject.

In various embodiments, disclosed herein is a method for predicting protective immunity against a virus in a subject that has been vaccinated with a vaccine, the method comprising detecting expression levels of multiple genes and/or gene products in a biological sample from the subject, wherein the multiple genes and/or gene products have been identified as a differentially expressed gene set associated with protective immunity against HIV or SIV and comprise at least 5, at least 6, at least 10, at least 20, at least 30, at least 40, at least 50, or 52 of the following genes: ALDOA, AOAH, AP2S1, APOBR, ARHGEF10L, ATF3, CD163, CEBPD, CLEC4A, CREB5, CSFIR, CSF3R, CST3, CTNNA1, DOCK5, DUSP6, EFHD2, ERMAP, FCGRT, GAA, GABARAPL1, GNAO, GRN, H2AFY, HBEGF, HSBP1, ICAM1, IRAK3, IL17RA, KIAA0513, KLF10, LST1, MAFB, MCL1, NAGA, NAMPT, NLRP3, OGFRL1, PSAP, PSTPIP1, RAB32, RGS10, RNF130, SIRPA, SLCO3A1, TBC1D8, TKT, TNFSF13, TRIB1, TYROBP, VIM, and WDFY3. The method further comprises calculating a first composite GES for the differentially expressed gene set in the biological sample from the subject, and calculating an average second composition GES for the differentially expressed gene set in a plurality of biological samples from subjects that have been vaccinated with the vaccine and are not immune to the virus, wherein a first composite GES that is greater than the average second composite GES indicates the subject that has been vaccinated with the candidate vaccine has protective immunity against the virus. In certain embodiments, the differentially expressed gene set comprises at least CREB5, DOCK5, ERMAP, IRAK3, OGFRL1, and RNF130. In certain embodiments, the method further comprises identifying the differentially expressed gene set. In certain embodiments, the method further comprises a step of adjusting treatment options for the subject based on whether the subject is indicated to have protective immunity against HIV or SIV.

In various embodiments, disclosed herein is a method for predicting effectiveness of a vaccine against HIV or SIV in a subject that has been vaccinated with the vaccine, the method comprising detecting expression levels of multiple genes and/or gene products in a biological sample from the subject, wherein the multiple genes and/or gene products have been identified as a differentially expressed gene set associated with efficacy of the vaccine and comprise at least 5, at least 10, at least 20, at least 30, at least 40, at least 50, at least 60, or 63 of the following genes: ADAM9, ALDH3B1, AMPD2, AP2S1, ARRB1, C5AR1, CAMKK2, CCR1, CD14, CD163, CD68, CEBPA, CSFIR, CST3, CTBP2, CTSD, DAPK1, DUSP6, FCGRT, FLVCR2, FZD1, GAA, GAS7, GRN, HMOX1, IL17RA, IMPA2, KIAA0513, LEPROT, LGALS2, LMNA, LRPAP1, MAFB, MPP1, MYOIF, NAGA, NPL, PGD, PKM2, PSAP, PTGER2, PXN, PYGL, QPCT, RAB20, RBM47, RNF130, SCARB2, SEMA4A, SERINC5, SIRPA, SIRPB1, SLC36A1, SLCO3A1, SORT1, SULT1A2, TBC1D8, TBX4S1, TLMP2, TKT, TYROBP, ZDHHC7, and ZNF467. The method further comprises calculating a first composite GES for the differentially expressed gene set in the biological sample from the subject, and calculating an average second composition GES for the differentially expressed gene set in a plurality of biological samples from subjects that have been vaccinated with the vaccine and are not immune to the virus, wherein a first composite GES that is greater than the average second composite GES indicates effectiveness of the vaccine. In certain embodiments, the differentially expressed gene set comprises at least SEMA4A, SLC36A1, SERINC5, IL17RA, CTSD, CD68, and GAA. In certain embodiments, the method further comprises identifying the differentially expressed gene set. In certain embodiments, the method further comprises a step of adjusting treatment options for the subject based on the predicted effectiveness of the vaccine in the subject.

In various embodiments, disclosed herein is a method for predicting protective immunity against a virus in a subject that has been vaccinated with a vaccine, the method comprising detecting expression levels of multiple genes and/or gene products in a biological sample from the subject, wherein the multiple genes and/or gene products have been identified as a differentially expressed gene set associated with protective immunity against HIV or SIV and comprise at least 5, at least 10, at least 20, at least 30, at least 40, at least 50, at least 60, or 63 of the following genes: ADAM9, ALDH3B1, AMPD2, AP2S1, ARRB1, C5AR1, CAMKK2, CCR1, CD14, CD163, CD68, CEBPA, CSFIR, CST3, CTBP2, CTSD, DAPK1, DUSP6, FCGRT, FLVCR2, FZD1, GAA, GAS7, GRN, HMOX1, IL17RA, IMPA2, KIAA0513, LEPROT, LGALS2, LMNA, LRPAP1, MAFB, MPP1, MYOIF, NAGA, NPL, PGD, PKM2, PSAP, PTGER2, PXN, PYGL, QPCT, RAB20, RBM47, RNF130, SCARB2, SEMA4A, SERINC5, SIRPA, SIRPB1, SLC36A1, SLCO3A1, SORT1, SULT1A2, TBC1D8, TBXAS1, TIMP2, TKT, TYROBP, ZDHHC7, and ZNF467. The method further comprises calculating a first composite GES for the differentially expressed gene set in the biological sample from the subject, and calculating an average second composition GES for the differentially expressed gene set in a plurality of biological samples from subjects that have been vaccinated with the vaccine and are not immune to the virus, wherein a first composite GES that is greater than the average second composite GES indicates the subject that has been vaccinated with the candidate vaccine has protective immunity against the virus. In certain embodiments, the differentially expressed gene set comprises at least SEMA4A, SLC36A1, SERINC5, IL17RA, CTSD, CD68, and GAA. In certain embodiments, the method further comprises identifying the differentially expressed gene set. In certain embodiments, the method further comprises a step of adjusting treatment options for the subject based on whether the subject is indicated to have protective immunity against HIV or SIV.

In certain embodiments, disclosed herein is a method for collecting data for evaluating protective immunity against a virus in a subject that has been vaccinated with a vaccine, the method comprising detecting expression of multiple genes and/or gene products in a biological sample from the subject, wherein the multiple genes and/or gene products have been identified as a differentially expressed gene set associated with protective immunity against HIV or SIV and comprise at least 3, at least 10, at least 15, at least 20, at least 25, at least 30, or 32 of the following genes: ACSL1, BHLHE40, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, OGFRL1, PHACTR2, RAB32, REEP4, RNF130, RRAGD, SDCBP, SIRPA, ST3GAL6, TKT, TNFRSF1B, TNFSF13, and VEGFA. The method further comprises calculating a first composite GES based on the expression levels of the genes, and calculating an average second composite GES based on expression levels of the genes in the differentially expressed gene set in a plurality of biological samples from subjects that have been vaccinated with the vaccine and are not immune to HIV or SIV. In certain embodiments, the differentially expressed gene set comprises at least BHLHE40, OGFRL1, and TNFSF13. In certain embodiments, the method further comprises identifying the differentially expressed gene set.

In certain embodiments, disclosed herein is a method for collecting data for evaluating protective immunity against a virus in a subject that has been vaccinated with a vaccine, the method comprising detecting expression of multiple genes in a biological sample from the subject, wherein the multiple genes have been identified as a differentially expressed gene set associated with protective immunity against HIV or SIV and comprise at least 5, at least 6, at least 10, at least 20, at least 30, at least 40, at least 50, at least 60, or 62 of the following genes: ACSL1, AHNAK, AOAH, ARHGEF10L, ARRB1, BHLHE40, CAMKK2, CD86, CD163, CDC42EP3, CEBPA, CEBPD, CLEC4A, CPD, CPPED1, CREB5, CREG1, CSF3R, CST1, CTNNA1, DAPK1, DOCK5, EMILIN2, ENO1, ERMAP, FAM105A, FBXL5, GLA, GNS, HBEGF, HEXB, HPSE, HSBP1, ID2, IRAK3, KIAA0513, LAMP2, LMNA, MAFB, MAPKAPK3, MICAL2, MYO1F, NFIL3, OGFRL1, PGD, PSAP, RAB27A, RNF130, RRAGD, RXRA, SCARB2, SDCBP, SEM44A, SLC31A2, SLC36A1, STX11, TBC1D8, TNFRSF1B, TNFSF13, VEGFA, VPS37C, and WDFY3. The method further comprises detecting expression of at least 5, at least 6, at least 10, at least 20, at least 30, at least 40, at least 50, at least 60, or 62 of the genes in the differentially expressed gene set in a biological sample from the subject; calculating a first composite GES based on the expression of the genes, and calculating an average second composite GES based on expression of the genes in the differentially expressed gene set in a plurality of biological samples from subjects that have been vaccinated with the vaccine and are not immune to HIV or SIV. In certain embodiments, the differentially expressed gene set comprises at least CREB5, DOCK5, ERMAP, IRAK3, OGFRL1, and RNF130. In certain embodiments, the method further comprises identifying the differentially expressed gene set.

In certain embodiments, disclosed herein is a method for collecting data for evaluating protective immunity against a virus in a subject that has been vaccinated with a vaccine, the method comprising detecting expression of multiple genes in a biological sample from the subject, wherein the multiple genes have been identified as a differentially expressed gene set associated with protective immunity against HIV or SIV and comprise at least 5, at least 6, at least 10, at least 20, at least 30, at least 40, at least 50, or 52 of the following genes: ALDOA, AOAH, AP2S1, APOBR, ARHGEF10L, ATF3, CD163, CEBPD, CLEC4A, CREB5, CSF1R, CSF3R, CST3, CTNNA1, DOCK5, DUSP6, EFHD2, ERMAP, FCGRT, GAA, GABARAPL1, GNAO, GRN, H2AFY, HBEGF, HSBPJ, ICAM1, IRAK3, IL17RA, KIAA0513, KLF10, LST1, MAFB, MCL1, NAGA, NAMPT, NLRP3, OGFRL1, PSAP, PSTPIP1, RAB32, RGS10, RNF130, SIRPA, SLCO3A1, TBC1D8, TKT, TNFSF13, TRIB1, TYROBP, VIM, and WDFY3. The method further comprises detecting expression of at least 5, at least 6, at least 10, at least 20, at least 30, at least 40, at least 50, or 52 of the genes in the differentially expressed gene set in a biological sample from the subject; calculating a first composite GES based on the expression of the genes, and calculating an average second composite GES based on expression of the genes in the differentially expressed gene set in a plurality of biological samples from subjects that have been vaccinated with the vaccine and are not immune to HIV or SIV. In certain embodiments, the differentially expressed gene set comprises at least CREB5, DOCK5, ERMAP, IRAK3, OGFRL1, and RNF130. In certain embodiments, the method further comprises identifying the differentially expressed gene set.

In certain embodiments of all aspects of the disclosure, gene set enrichment analysis (GSEA) may be used to identify classes of genes that may have an association with infection status post challenge. GSEA allows for the interpretation of gene expression data, focusing on gene sets, or groups of genes that share a common function, location, or regulation. Gene sets are available in a searchable format in the electronic Molecular Signatures Database (MSigDB). Genes from a sample may be ranked by differential gene expression, comparing protected to infected animals, and used to screen for enriched gene sets in the MSigDB. Normalized enrichment scores computed for each gene set may reflect the degree to which a gene set correlates with protection.

Differentially expressed gene sets may be sorted, for example, by functions such as apoptosis, differentiation, growth factor, metabolism, and cell signaling and proliferation. In a differentially expressed gene set disclosed herein, for example, DAPK1, EMILIN2, NFIL3, RNF130, and TNRSF1B may be associated with apoptosis. BHLHE40, CREG1, and ERMAP may be associated with differentiation. GAS7, OGFRL1, and VEGFA may be associated with growth factor. ACSL1, GLA, and TKT may be associated with metabolism. CD4, CDC42EP3, CDC42EP4, CREB5, DOCK5, IRAK3, LAMP2, LMNA, MGST2, NLRP3, PHACTR2, RAB32, REEP4, RRAGD, SDCBP, SIRPA, ST3GAL6, and TNFSF13 may be associated with cell signaling and/or proliferation.

In certain embodiments of all aspects of the disclosure, enrichment of a gene set may be greatest at a specific time point post-vaccination, such as, for example, one week, two weeks, one month, or two months post-vaccination. In certain embodiments of all aspects of the disclosure, enrichment of a gene set may still be present for a given period of time post-vaccination, such as, for example, 2 months, 4 months, 6 months, 1 year, 2 years, 5 years, 10 years post-vaccination or for the remainder of the lifetime of the vaccinated subject.

The differentially expressed gene sets disclosed herein may be associated with other immune responses in addition to the GES. Other immune responses that be associated with the differentially expressed gene sets include, for example, higher levels of antibody-dependent cellular phagocytosis (ADCP), antibody-dependent complement deposition (ADCD), antibody-dependent cellular cytotoxicity (ADCC), antibody Fc polyfunctionality, IgG breadth, tier 1 Nab titers, percent tier 2 neutralization, and pol-specific cells, as well as increased cellular responses to Gag, gp140, CD107a, interferon-γ, and chemokine CCL4.

As discussed above, in certain exemplary embodiments of all aspects of the disclosure, a differentially expressed gene set may comprise at least BHLHE40, OGFRL1, and TNFSF13. BHLHE40 has been shown to suppress IL10; OGFRL1 is a paralog gene for the opioid growth factor, and TNFSF13 belongs to the tumor necrosis factor ligand super family. TNFSF13 is also known as APRIL (a proliferation-inducing ligand) and is a ligand for TNFRSF17/BCMA or TNFSF13B/TACI, both of which are involved in numerous B-cell functions including class switching, affinity maturation, and antibody production. TNFRSF17 expression has been shown to correlate with increased magnitude of vaccine-induced antibody titers against influenza and yellow fever. TNFSF13 is generally produced by innate immune cells, but can also be induced in activated B cells. DNA vaccines encoding TNFSF13 as a molecular adjuvant elicit antibody production with increased HIV-1 neutralization activity. TNFSF13 also increases the longevity of humoral immunity and protection to influenza virus infection.

Methods of Vaccination

Another aspect is directed to methods of vaccinating a subject with a vaccine. For example, a method of a vaccinating a subject with an HIV or SIV vaccine is provided, the method comprising administering the HIV or SIV vaccine to the subject, wherein prior to the administering step, the subject has been identified as having protective immunity against HIV or SIV using the methods or predicting protective immunity disclosed herein. Also provided is a method of a vaccinating a subject with an HIV or SIV vaccine, the method comprising administering the HIV or SIV vaccine to the subject, wherein prior to the administering step, the vaccine has been identified as an effective vaccine in the subject using the methods of predicting vaccine effectiveness disclosed herein.

Detecting Gene Expression

As used herein, measuring or detecting the expression of genes or nucleic acids comprises measuring or detecting any nucleic acid transcript (e.g., mRNA or cDNA) corresponding to the gene of interest or the protein encoded thereby. The presence or absence of a gene may be detected by measuring or detecting the expression of a gene or nucleic acids, for example if the gene or nucleic acids are not detected, or if the measurement of the expression of the gene or nucleic acid falls below a threshold level, the gene or nucleic acids may be determined to be absent. Likewise, if the gene or nucleic acids are detected, or if the measurement of the expression of the gene or nucleic acid falls above a threshold level, the gene or nucleic acids may be determined to be present. If a gene is associated with more than one mRNA transcript or isoform, the expression of the gene can be measured or detected by measuring or detecting one or more of the mRNA transcripts of the gene, or all of the mRNA transcripts associated with the gene.

Typically, gene expression can be detected or measured on the basis of mRNA or cDNA levels, although protein levels also can be used when appropriate. Any quantitative or qualitative method for measuring mRNA levels, cDNA, or protein levels can be used. Suitable methods of detecting or measuring mRNA or cDNA levels include, for example, Northern Blotting, microarray analysis, RNA-sequencing, or a nucleic acid amplification procedure, such as reverse-transcription PCR (RT-PCR) or real-time RT-PCR, also known as quantitative RT-PCR (qRT-PCR). Such methods are well known in the art. See e.g., Sambrook et al., Molecular Cloning: A Laboratory Manual. 4^{th} Ed., Cold Spring Harbor Press, Cold Spring Harbor, N.Y., 2012. Other techniques include digital, multiplexed analysis of gene expression, such as the nCounter® (NanoString Technologies, Seattle, Wash.) gene expression assays, which are further described in US20100112710 and US20100047924.

Detecting a nucleic acid of interest generally involves hybridization between a target (e.g., mRNA, cDNA, or genomic DNA) and a probe. The nucleic acid sequences of the genes described herein are known. Therefore, one of skill in the art can readily design hybridization probes for detecting those genes. See, e.g. Sambrook et al., Molecular Cloning: A Laboratory Manual, 4^{th} Ed., Cold Spring Harbor Press, Cold Spring Harbor, N.Y., 2012, For example, polynucleotide probes that specifically bind to the mRNA transcripts of the genes described herein (or cDNA synthesized therefrom) can be created using the nucleic acid sequences of the mRNA or cDNA targets themselves by routine techniques (e.g., PCR or synthesis). As used herein, the term “fragment” means a part or portion of a polynucleotide sequence comprising about 10 or more contiguous nucleotides, about 15 or more contiguous nucleotides, about 20 or more contiguous nucleotides, about 30 or more, or even about 50 or more contiguous nucleotides. In certain embodiments, the polynucleotide probes will comprise 10 or more nucleic acids, 20 or more, 50 or more, or 100 or more nucleic acids. In order to confer sufficient specificity, the probe may have a sequence identity to a complement of the target sequence of about 90% or more, such as about 95% or more (e.g., about 98% or more or about 99% or more) as determined, for example, using the well-known Basic Local Alignment Search Tool (BLAST) algorithm (available through the National Center for Biotechnology Information (NCBI), Bethesda, Md.).

Each probe may be substantially specific for its target, to avoid any cross-hybridization and false positives. An alternative to using specific probes is to use specific reagents when deriving materials from transcripts (e.g., during cDNA production, or using target-specific primers during amplification). In both cases specificity can be achieved by hybridization to portions of the targets that are substantially unique within the group of genes being analyzed, for example hybridization to the polyA tail would not provide specificity. If a target has multiple splice variants, it is possible to design a hybridization reagent that recognizes a region common to each variant and/or to use more than one reagent, each of which may recognize one or more variants.

Stringency of hybridization reactions is readily determinable by one of ordinary skill in the art, and generally is an empirical calculation dependent upon probe length, washing temperature, and salt concentration. In general, longer probes may require higher temperatures for proper annealing, while shorter probes may require lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so.

In some embodiments of all aspects of the disclosure, RNA-sequencing (RNA-seq) is used. As used herein, RNA-seq, also called Whole Transcriptome Shotgun Sequencing, refers to any of a variety of high-throughput sequencing techniques used to detect the presence and quantity of RNA transcripts in real time. See Wang, Z., M. Gerstein, and M. Snyder, RNA-Seq: a revolutionary tool for transcriptomics, NAT REV GENET, 2009. 10(1): p. 57-63. RNA-seq can be used to reveal a snapshot of a sample's RNA from a genome at a given moment in time. In certain embodiments of all aspects of the disclosure, RNA is converted to cDNA fragments via reverse transcription prior to sequencing, and, in certain embodiments, RNA can be directly sequenced from RNA fragments without conversion to cDNA. Adaptors may be attached to the 5′- and/or 3′-ends of the fragments, and the RNA or cDNA may optionally be amplified, for example by PCR. The fragments are then sequenced using high-throughput sequencing technology, such as, for example, those available from Roche (e.g., the 454 platform), Illumina, Inc., and Applied Biosystem (e.g., the SOLiD system).

In some embodiments of all aspects of the disclosure, microarray analysis or a PCR-based method is used. In this respect, measuring the expression of nucleic acids in a biological sample can comprise, for instance, contacting a sample, such as a sample comprising lymphocytes, with polynucleotide probes specific to the genes of interest, or with primers designed to amplify a portion of the genes of interest, and detecting binding of the probes to the nucleic acid targets or amplification of the nucleic acids, respectively. Detailed protocols for designing PCR primers are known in the art. See e.g., Sambrook et al., Molecular Cloning: A Laboratory Manual, 4^{th} Ed., Cold Spring Harbor Press, Cold Spring Harbor, N.Y., 2012. Similarly, detailed protocols for preparing and using microarrays to analyze gene expression are known in the art and described herein.

Alternatively or additionally, expression levels of genes can be determined at the protein level, meaning that levels of proteins encoded by the genes discussed herein are measured. Several methods and devices are known for determining levels of proteins including immunoassays, such as described, for example, in U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,458,852; and 5,480,792, each of which is hereby incorporated by reference in its entirety. These assays may include various sandwich, competitive, or non-competitive assay formats, to generate a signal that is related to the presence or amount of a protein of interest. Any suitable immunoassay may be utilized, for example, lateral flow, enzyme-linked immunoassays (ELISA), radioimmunoassays (RIAs), competitive binding assays, and the like. Numerous formats for antibody arrays have been described. Such arrays may include different antibodies having specificity for different proteins intended to be detected. For example, at least 100 different antibodies are used to detect 100 different protein targets, each antibody being specific for one target. Other ligands having specificity for a particular protein target can also be used, such as the synthetic antibodies disclosed in WO 2008/048970, which is hereby incorporated by reference in its entirety. Other compounds with a desired binding specificity can be selected from random libraries of peptides or small molecules. U.S. Pat. No. 5,922,615, which is hereby incorporated by reference in its entirety, describes a device that uses multiple discrete zones of immobilized antibodies on membranes to detect multiple target antigens in an array. Microtiter plates or automation can be used to facilitate detection of large numbers of different proteins.

Samples

The methods described herein involve analysis of gene expression profiles in biological samples obtained from subjects, such as subjects who have been previously vaccinated with a vaccine or candidate vaccine, subjects who are known to be immune to a virus of interest, subjects who may be at an increased risk of virus acquisition, and subjects who are not infected with HIV or SIV. In certain embodiments of all aspects of the disclosure, the sample may comprise blood and blood cells. In certain embodiments of all aspects of the disclosure, the sample may comprise cells from a blood sample. In certain embodiments of all aspects of the disclosure, the sample comprises peripheral blood mononuclear cells, such as, for example, lymphocytes and/or myeloid cells. Nucleic acids or polypeptides may be isolated from the sample prior to detecting gene expression. In one embodiment of all aspects of the disclosure, the biological sample comprises a blood sample. The methods disclosed herein can be used with biological samples collected from a variety of mammals, such as human and non-human primates, and in certain embodiments, the methods disclosed herein may be used with biological samples obtained from a human subject. In certain embodiments of all aspects of the disclosure, the samples may be cryopreserved prior to detecting gene expression.

Controls

In certain embodiments of all aspects of the disclosure, the control may be any suitable reference that allows evaluation of the expression level of the genes in the biological sample as compared to the expression of the same genes in a sample comprising control cells. In certain embodiments of all aspects of the disclosure, the control cells may be cells from a subject or subjects that have not been vaccinated with the vaccine or candidate vaccine, and in certain embodiments, the control cells may be cells from a subject or subjects that are known not to have been exposed to a particular virus, such as HIV or SIV. The control can be a sample that is analyzed simultaneously or sequentially with the test sample. In certain embodiments of all aspects of the disclosure, the control can be the average expression level of the genes of interest in a pool of samples from subjects known to have not been vaccinated and/or exposed to the virus. In certain embodiments of all aspects of the disclosure, the control can be embodied, for example, in a pre-prepared microarray used as a standard or reference, or in data that reflects the expression profile of relevant genes in a sample or pool of samples from subjects known to have not been vaccinated and/or exposed to the virus, such as might be part of an electronic database or computer program.

Overexpression and decreased expression (under-expression) of a gene can be determined by any suitable method, such as by comparing the expression of the genes in a test sample with a control gene or threshold value. In certain embodiments of all aspects of the disclosure, the control gene is one or more housekeeping genes, such as ACTB, GAPDH, HMBS, GUSB, or RPLPO, that can be used to normalize gene expression levels. Regardless of the method used, overexpression and under-expression can be defined as any level of expression greater than or less than the level of expression of a control gene or threshold value, such as the normalized gene expression value. By way of further illustration, overexpression can be defined as expression that is at least about 1.2-fold, 1.5-fold, 2-fold, 2.5-fold, 4-fold, 5-fold, 10-fold, 20-fold, 50-fold, 100-fold higher or even greater expression as compared to tissue control gene or threshold value, and under-expression can similarly be defined as expression that is at least about 1.2-fold, 1.5-fold, 2-fold, 2.5-fold, 4-fold, 5-fold, 10-fold, 20-fold, 50-fold, 100-fold lower or even lower expression as compared to tissue control gene or threshold value.

Arrays

A convenient way of measuring RNA transcript levels for multiple genes in parallel is to use an array (also referred to as microarrays in the art). A useful array may include multiple polynucleotide probes (such as DNA) that are immobilized on a solid substrate (e.g., a glass support such as a microscope slide, or a membrane) in separate locations (e.g., addressable elements) such that detectable hybridization can occur between the probes and the transcripts to indicate the amount of each transcript that is present. The arrays disclosed herein can be used in methods of detecting the expression of a desired combination of genes, which combinations are discussed herein.

In one embodiment of all aspects of the disclosure, the array comprises (a) a substrate and (b) at least 3, such as at least 5, at least 8, at least 10, at least 15, at least 20, at least 25, at least 30, or 32 different addressable elements that each comprise at least one polynucleotide probe for detecting the expression of an mRNA transcript (or cDNA synthesized from the mRNA transcript) that is specific for one of the genes in the gene signature, such that the array can be used to simultaneously detect the expression of these at least 3, at least 5, at least 8, at least 10, at least 15, at least 20, at least 25, at least 30, or 32 genes.

In certain embodiments of all aspects of the disclosure, the array further comprises one or more different addressable elements comprising at least one oligonucleotide probe for detecting the expression of an mRNA transcript (or cDNA synthesized from the mRNA transcript) of a control gene.

As used herein, the term “addressable element” means an element that is attached to the substrate at a predetermined position and specifically binds a known target molecule, such that when target-binding is detected (e.g., by fluorescent labeling), information regarding the identity of the bound molecule is provided on the basis of the location of the element on the substrate. Addressable elements are “different” for the purposes of the present disclosure if they do not bind to the same target gene. The addressable element comprises one or more polynucleotide probes specific for an mRNA transcript of a given gene, or a cDNA synthesized from the mRNA transcript. The addressable element can comprise more than one copy of a polynucleotide or can comprise more than one different polynucleotide, provided that all of the polynucleotides bind the same target molecule. Where a gene is known to express more than one mRNA transcript, the addressable element for the gene can comprise different probes for different transcripts, or probes designed to detect a nucleic acid sequence common to two or more (or all) of the transcripts. Alternatively, the array can comprise an addressable element for the different transcripts. The addressable element also can comprise a detectable label, suitable examples of which are well known in the art.

The array can comprise addressable elements that bind to mRNA or cDNA other than that of the genes in the gene signature. However, an array capable of detecting a vast number of targets (e.g., mRNA or polypeptide targets), such as arrays designed for comprehensive expression profiling of a cell line, chromosome, genome, or the like, may not be economical or convenient for collecting data to use in diagnosing and/or prognosing cancer. Thus, the array typically comprises no more than about 1000 different addressable elements, such as no more than about 500 different addressable elements, no more than about 250 different addressable elements, or even no more than about 100 different addressable elements, such as about 75 or fewer different addressable elements, about 62 or fewer different addressable elements, about 60 or fewer different addressable elements, about 52 or fewer different addressable elements, about 50 or fewer different addressable elements, about 40 or fewer different addressable elements, about 32 or fewer different addressable elements, about 30 or fewer different addressable elements, about 20 or fewer different addressable elements, about 15 or fewer, about 10 or fewer, about 8 or fewer, about 6 or fewer, about 5 or fewer, or about 3 different addressable elements.

It is also possible to distinguish these diagnostic arrays from the more comprehensive genomic arrays and the like by limiting the number of polynucleotide probes on the array. For example, the array may have polynucleotide probes for no more than 1000 genes immobilized on the substrate. In other embodiments, the array has oligonucleotide probes for no more than 500, no more than 250, no more than 100, no more than 75, no more than 60, or no more than 50 genes. In certain embodiments, the array has oligonucleotide probes for no more than 40 genes, and in certain embodiments, the array has oligonucleotide probes for no more than 30 genes, no more than 20 genes, or no more than 15 genes.

The substrate can be any rigid or semi-rigid support to which polynucleotides can be covalently or non-covalently attached. Suitable substrates include membranes, filters, chips, slides, wafers, fibers, beads, gels, capillaries, plates, polymers, microparticles, and the like. Materials that are suitable for substrates include, for example, nylon, glass, ceramic, plastic, silica, aluminosilicates, borosilicates, metal oxides such as alumina and nickel oxide, various clays, nitrocellulose, and the like.

The polynucleotides of the addressable elements (also referred to as “probes”) can be attached to the substrate in a pre-determined 1- or 2-dimensional arrangement, such that the pattern of hybridization or binding to a probe is easily correlated with the expression of a particular gene. Because the probes are located at specified locations on the substrate (i.e., the elements are “addressable”), the hybridization or binding patterns and intensities create a unique expression profile, which can be interpreted in terms of expression levels of particular genes and can be correlated with vaccine efficacy in accordance with the methods described herein.

The array can comprise other elements common to polynucleotide arrays. For instance, the array also can include one or more elements that serve as a control, standard, or reference molecule, such as a housekeeping gene or portion thereof, to assist in the normalization of expression levels or the determination of nucleic acid quality and binding characteristics, reagent quality and effectiveness, hybridization success, analysis thresholds and success, etc. These other common aspects of the arrays or the addressable elements, as well as methods for constructing and using arrays, including generating, labeling, and attaching suitable probes to the substrate, consistent with the invention are well-known in the art. Other aspects of the array are as described with respect to the methods disclosed herein.

An array can also be used to measure protein levels of multiple proteins in parallel. Such an array comprises one or more supports bearing a plurality of ligands that specifically bind to a plurality of proteins, wherein the plurality of proteins comprises no more than 500, no more than 250, no more than 100, no more than 75, no more than 62, no more than 60, no more than 52, no more than 50, no more than 40, no more than 32, no more than 30, no more than 20, no more than 15, no more than 10, no more than 8, no more than 6, no more than 5, or no more than 3 different proteins. The ligands are optionally attached to a planar support or beads. In one embodiment, the ligands are antibodies. The proteins that are to be detected using the array correspond to the proteins encoded by the nucleic acids of interest, as described above, including the specific gene expression profiles disclosed. Thus, each ligand (e.g. antibody) is designed to bind to one of the target proteins (e.g., polypeptide sequences encoded by the genes disclosed herein). As with the nucleic acid arrays, each ligand may be associated with a different addressable element to facilitate detection of the different proteins in a sample.

In certain embodiments of all aspects of the disclosure, disclosed herein are methods of obtaining a gene expression profile in a biological sample, such as a blood sample, the method comprising: a) incubating an array as disclosed herein with the biological sample; and b) measuring the expression level of the genes of interest.

Compositions and Kits

The polynucleotide probes and/or primers or antibodies or polypeptide probes that can be used in the methods described herein can be assembled into kits. Thus, one embodiment is directed to a kit for evaluating efficacy and/or effectiveness of an HIV or an SIV vaccine. In some aspects, a kit is disclosed for predicting protective immunity for subjects following HIV or SIV vaccination, or a kit for collecting data for evaluating such protective immunity.

In certain embodiments of all aspects of the disclosure, provided herein is a kit for measuring expression levels of a plurality of genes comprising a plurality of polynucleotide probes for detecting at least 3, such as at least 5, 6, at least 8, at least 10, at least 15, at least 20, at least 25, at least 30, 32, at least 40, at least 50, 52, at least 60, 62, or 63 genes in a gene signature, wherein the plurality of probes contains polynucleotide probes for no more than 500, 250, 100, 75, 60, 50, 40, 30, 25, 20, 15, 10, 8, or 5 genes. In one embodiment, the kit comprises at least one oligonucleotide probe for detecting the expression of a control gene. All of the polynucleotide probes described herein may be optionally labeled. Such labeled polynucleotide probes are not naturally occurring.

In some embodiments of all aspects of the disclosure, the kit may optionally include polynucleotide primers for amplifying a portion of the mRNA transcripts from at least 3, at least 5, at least 6, at least 10, at least 15, at least 20, at least 25, at least 30, at least 32, at least 40, at least 50, at least 52, at least 60, at least 62, or at least 63 of the genes in the gene signature.

In certain embodiments of all aspects of the disclosure, disclosed herein is a kit for measuring expression levels of a plurality of genes, the kit comprising a plurality of probes for detecting at least 3, at least 5, at least 8, at least 10, at least 15, at least 20, at least 25, at least 30, or 32 of the following genes: ACSL1, BHLHE40, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, OGFRL1, PHACTR2, RAB32, REEP4, RNF130, RRAGD, SDCBP, SIRPA, ST3GAL6, TKT, TNFRSF1B, TNFSF13, and VEGFA, wherein the plurality of probes contains probes for detecting no more than 500 different genes. In certain embodiments, the kit comprises a plurality of probes for detecting at least BHLHE40, OGFRL1, and TNFSF13. In certain embodiments, the plurality of probes contains probes for detecting no more than 250, 100, 75, 60, 50, 40, 32, 30, 25, 20, 15, 10, 8, or 3 different genes.

In certain embodiments of all aspects of the disclosure, disclosed herein is a kit for measuring expression of a plurality of genes, the kit comprising a plurality of probes for detecting at least 5, at least 6, at least 10, at least 20, at least 30, at least 40, at least 50, at least 60, or 62 of the following genes: ACSL1, AHNAK, AOAH, ARHGEF10L, ARRB1, BHLHE40, CAMKK2, CD86, CD163, CDC42EP3, CEBPA, CEBPD, CLEC4A, CPD, CPPED1, CREB5, CREG1, CSF3R, CST1, CTNNA1, DAPK1, DOCK5, EMILIN2, ENO1, ERMAP, FAM105A, FBXL5, GLA, GNS, HBEGF, HEXB, HPSE, HSBP1, ID2, IRAK3, KIAA0513, LAMP2, LMNA, MAFB, MAPKAPK3, MICAL2, MYOIF, NFIL3, OGFRL1, PGD, PSAP, RAB27A, RNF130, RRAGD, RXRA, SCARB2, SDCBP, SEMA4A, SLC31A2, SLC36AJ, STX11, TBC1D8, TNFRSF1B, TNFSF13, VEGFA, VPS37C, and WDFY3, wherein the plurality of probes contains probes for detecting no more than 500 different genes. In certain embodiments, the kit comprises a plurality of probes for detecting at least CREB5, DOCK5, ERMAP, IRAK3, OGFRL1, and RNF130. In certain embodiments, the plurality of probes contains probes for detecting no more than 250, 100, 75, 62, 60, 50, 40, 30, 25, 20, 15, 10, 8, 6, or 5 different genes.

In certain embodiments of all aspects of the disclosure, disclosed herein is a kit for measuring expression levels of a plurality of genes, the kit comprising a plurality of probes for detecting at least 5, at least 6, at least 10, at least 20, at least 30, at least 40, at least 50, or 52 of the following genes: ALDOA, AOAH, AP2S, APOBR, ARHGEF10L, ATF3, CD163, CEBPD, CLEC4A, CREB5, CSF1R, CSF3R, CST3, CTNNA1, DOCK5, DUSP6, EFHD2, ERMAP, FCGRT, GAA, GABARAPL1, GNAO, GRN, H2AFY, HBEGF, HSBP1, ICAM1, IRAK3, IL17RA, KIAA0513, KLF10, LST1, MAFB, MCL1, NAGA, NAMPT, NLRP3, OGFRL1, PSAP, PSTPIP1, RAB32, RGS10, RNF130, SIRPA, SLCO3A1, TBC1D8, TKT, TNFSF13, TRIB1, TYROBP, VIM and WDFY3, wherein the plurality of probes contains probes for detecting no more than 500 different genes. In certain embodiments, the kit comprises a plurality of probes for detecting at least CREB5, DOCK5, ERMAP, IRAK3, OGFRL1, and RNF130. In certain embodiments, the plurality of probes contains probes for detecting no more than 250, 100, 75, 60, 52, 50, 40, 30, 25, 20, 15, 10, 8, 6, or 5 different genes.

In certain embodiments of all aspects of the disclosure, disclosed herein is a kit for measuring expression levels of a plurality of genes, the kit comprising a plurality of probes for detecting at least 5, at least 6, at least 10, at least 20, at least 30, at least 40, at least 50, at least 60, or 63 of the following genes: ADAM9, ALDH3B1, AMPD2, AP2S1, ARRB1, C5AR1, CAMKK2, CCR1, CD14, CD163, CD68, CEBPA, CSFIR, CST3, CTBP2, CTSD, DAPK1, DUSP6, FCGRT, FLVCR2, FZDJ, GAA, GAS7, GRN, HMOX1, IL17RA, IMPA2, KIAA0513, LEPROT, LGALS2, LMNA, LRPAP1, MAFB, MPP1, MYO1F, NAGA, NPL, PGD, PKM2, PSAP, PTGER2, PXN, PYGL, QPCT, RAB20, RBM47, RNF130, SCARB2, SEMA4A, SERINC5, SIRPA, SIRPB1, SLC36A1, SLCO3A1, SORT1, SULT1A2, TBC1D8, TBXAS1, TMP2, TKT, TYROBP, ZDHHC7, and ZNF467, wherein the plurality of probes contains probes for detecting no more than 500 different genes. In certain embodiments, the kit comprises a plurality of probes for detecting at least SEMA4A, SLC36A1, SERINC5, IL17RA, CTSD, CD68, and GAA. In certain embodiments, the plurality of probes contains probes for detecting no more than 250, 100, 75, 63, 60, 50, 40, 30, 25, 20, 15, 10, 8, 6, or 5 different genes.

The kit for measuring expression of a plurality of genes may also comprise antibodies. For example, the kit may comprise a plurality of antibodies for detecting at least 3, at least 5, at least 6, at least 8, at least 10, at least 15, at least 20, at least 25, at least 30, at least 32, at least 40, at least 50, at least 52, at least 60, at least 62, or 63 of the polypeptides encoded by genes in the gene signature, wherein the plurality of antibodies contains antibodies for no more than 500, 250, 100, 75, 63, 62, 60, 52, 50, 40, 32, 30, 25, 20, 15, 10, 8, 6, 5, or 3 polypeptides.

As noted above, the polynucleotide or polypeptide probes and antibodies described herein may be optionally labeled with a detectable label. Any detectable label used in conjunction with probe or antibody technology, as known by one of ordinary skill in the art, can be used. As described herein, the labelled polynucleotide probes or labelled antibodies are not naturally occurring molecules; that is the combination of the polynucleotide probe coupled to the label or the antibody coupled to the label do not exist in nature. In certain embodiments, the probe or antibody is labeled with a detectable label selected from the group consisting of a fluorescent label, a chemiluminescent label, a quencher, a radioactive label, biotin, mass tags and/or gold.

In one embodiment, a kit includes instructional materials disclosing methods of use of the kit contents in a disclosed method. The instructional materials may be provided in any number of forms, including, but not limited to, written form (e.g., hardcopy paper, etc.), in an electronic form (e.g., computer diskette or compact disk) or may be visual (e.g., video files). The kits may also include additional components to facilitate the particular application for which the kit is designed. Thus, for example, the kits may additionally include other reagents routinely used for the practice of a particular method, including, but not limited to buffers, enzymes, labeling compounds, and the like. Such kits and appropriate contents are well known to those of skill in the art. The kit can also include a reference or control sample. The reference or control sample can be a biological sample or a data base.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

Unless indicated otherwise in these Examples, the methods involving commercial kits were done following the instructions of the manufacturers.

### Example 1. Differentially Expressed Gene Sets and Gene Expression Scores

Samples

Whole transcriptome profiling of bulk sorted lymphocytes isolated from peripheral blood of 21 rhesus monkeys from the Ad26/gp140 arm of the SIV (Barouch I) and SHIV (Barouch II) challenge preclinical trials was performed by next-generation sequencing (NGS) of RNA. Total RNA was extracted from four sorted lymphocyte populations of CD4 T cells, CD8 T cells, NK cells and B cells. RNA-Seq data was generated from time points post vaccination but prior to challenge in the Ad26/gp140 arm from both NHP studies.

Cryopreserved peripheral blood mononuclear cells (PBMCs) from 10 rhesus monkeys from the Barouch I study were obtained. Likewise, PBMCs from 32 rhesus monkeys from the Barouch II study were obtained. Monkeys were selected from the vaccine arms of those studies where partial efficacy was observed, namely the Ad26/gp140 arm of the Barouch I SIV challenge study (N=10) and the Ad26/gp140 (N=11), Ad26/Ad26+gp140 (N=12) and Ad26/MVA+gp140 (N=9) arms of the Barouch II SHIV study.

Antibody-Dependent Cellular Phagocytosis (ADCP) Assay

A THP-1 phagocytosis assay was performed. THP-1 cells were purchased from ATCC and cultured as recommended. Recombinant vaccine matched gp140 (32H) was used to saturate the binding sites on 1 μm fluorescent neutravidin beads (Invitrogen) overnight at 4° C. Excess antigen was removed by washing the pelleted beads, which were then incubated with patient antibody samples for 2 hours at 37° C. Following opsonization, THP-1 cells were added, and the cells were incubated overnight to allow phagocytosis. The cells were then fixed, and bead uptake was measured via flow cytometry on a BD FACScan equipped with high-throughput sampler. Phagocytic scores represent iMFI values (integrated MFI: frequency×MFI). Each antibody sample was tested over a range of concentrations (0.1-100 μg/ml).

ADCP was measured as described in Ackerman et al., A robust, high-throughput assay to determine the phagocytic activity of clinical antibody samples, J Immunol Methods 2011; 366, 8-19. Briefly, gp120 CRFO1_AE (CM235, Immune Technology, NY, USA) was biotinylated at a biotin to antigen ratio of 50 following manufacturer's instructions (Thermo Scientific), and excess biotin was removed using Zeba desalting columns (Thermo Scientific). Biotinylated antigen was then incubated with yellow-green strepavidin-fluorescent beads (Molecular Probes) for 2 h at 37° C. 10 μl of a 100-fold dilution of beads-antigen was incubated 2 h at 37° C. with 100 μl of a 200-fold dilution of plasma samples before addition of 20,000 THP-1 cells per well (MilliporeSigma, Burlington, Mass., USA). For negative control, beads-antigen were incubated with plasma samples from HIV-uninfected subjects. After 18 h incubation at 37° C., the cells were fixed with 2% formaldehyde solution (Tousimis, Rockville Md. USA) and fluorescence was evaluated on a LSRII (BD Bioscience). The phagocytic score was calculated by multiplying the percentage of bead-positive cells by the geo mean fluorescence intensity of the bead-positive cells and dividing by 10⁴.

Flow Cytometry

Cells were thawed and stained with a cocktail of antibodies including CD3 Alexa Fluor 700 (clone SP34-2), CD4 Brilliant Violet 605 (clone OKT4), CD8 Brilliant Violet 785 (clone RPA-T8), CD14 Pacific Blue (clone M5E2), CD16 PE-Cy7 (clone 3G8), CD20 FITC (clone 2H7), NKG2A PE (clone Z199), and Live/Dead Aqua viability dye to differentiate lymphocyte populations. The stained cells were analyzed and sorted using a BD FACS Aria II, producing pure bulk populations of CD4+ T cells, CD8+ T cells, NK cells, and B cells.

RNA Extraction and Sequencing

Total RNA was extracted from sorted lymphocytes in a two-step approach using the Single Cell RNA Purification kit (Norgen Biotek Corp, Ontario, Canada). Post-sorted cells were immediately pelleted, lysed, and stored in lysis solution at −80° C. The extraction process was continued per manufacturer's instructions after thawing and ethanol addition. RNA integrity number (RIN) scores and concentrations of the purified RNA were determined with the RNA 6000 Pico kit and the 2100 Bioanalyzer (both Agilent, Santa Clara, Calif.). Next Generation Sequencing (NGS) RNA-Seq was performed using SMART-Seq® technology. RNA fragmentation, first-strand cDNA synthesis, sample-specific adaptor/index addition, nbosomal cDNA depletion, and fragment amplification were performed in 96-well plates with 2.5 ng RNA inputs using the SMARTer Stranded Total RNA-Seq Pico Input Mammalian kit (Takara Bio Inc, Kusatsu, Japan) per manufacturer's instructions, but with spike-ins of exogenous control RNA (ERCC ExFold RNA Spike-in Mixes 1 or 2; ThermoFisher Scientific, Waltham, Mass.). ERCC was added to each sample to a final dilution of 1:260,000 in the initial 13 μl volume set-up of the SMARTer kit. Final library PCR amplifications were 13 cycles.

Quantitation of amplified cDNA libraries was performed by NGS-based MiSeq quantitation. Initial concentrations for each sample library were determined using the Qubit® dsDNA HS kit with the Qubit® Fluorometer (both ThermoFisher Scientific), while QC confirmations and fragment sizes of random samples were determined with the High Sensitivity DNA chip (Agilent). A portion of each sample was diluted to 2 nM in Resuspension Buffer prior to pooling 3 μl of each dilution into a collective sample pool. This pool was sequenced to determine the percent of total reads assigned to each sample, using a 50 cycle MiSeq® Reagent Kit v2 and MiSeq® instrument (both Illumina; San Diego, Calif.) per manufacturer's instructions for single-read output, with final input concentrations of 8 pM, and no PhiX. After analyzing the percent of total reads assigned to each sample, 2 nM sample dilutions were made from the original sample plate using different volumes as necessary to adjust for any deviations from the expected read percentage.

NGS was performed on pooled fractions with either HiSeq® 2500 (with TruSeq® PE Cluster cBot v3, and 200-cycle TruSeq® SBS v3 kits), or NextSeq® (with the 300-cycle NextSeq® 500 High Output v2 kit) instruments (all Illumina) per manufacturer's instructions for paired-end output targeting 50 Million reads per sample. For the HiSeq® approach, 8.5 pM of the diluted denatured cDNA from the pooled product spiked with 1% PhiX was loaded into flow cells. Starting input for the NextSeq® approach was 30 μl of a 150 pM dilution of the pooled product, followed by denaturation, neutralization, dilution, 1% PhiX spike-in, and loading.

Sequence Alignment and Expression Analysis

Paired-End (PE) sequencing data generated from Illumina HiSeq® and NextSeq® was converted to fastq, and adapters masked using bcl2fastq v2.17.1.14 software (Illumina). The raw fastq files were examined for quality using FastQC v0.11.5, and reads with quality scores (Q) of <30 were removed using Trimmomatic v0.36. Reads passing filter and with minimum length of 50 base pairs were retained. Reads passing filter were aligned to the non-human primate (NHP) (Macaca mulatta, NCBI version Mmul 8.0.1) genome assembly using HiSat2 v2.1.07. Gene expression quantification was performed using HTSeq-count v0.9.18 and trimmed mean of M-values (TMM) based normalization of the read counts were carried out using Limma and EdgeR v3.18.1 packages on the RStudio GUI v1.1.419 in a R v3.4 statistical software environment. Genes were filtered based on expression within a cell subset if ≥50% of the samples within a comparison group had normalized read counts per million ≥1. On average a total of 11286 genes were expressed per animal.

Microarray Data

Published normalized log transformed microarray data from two vaccine studies, DNA-SIV/ALVAC+gp120 and ALVAC-SIV/gp120, were downloaded from the GEO database (http://www.ncbi.nlm.nih.gov/geo/) having accession codes GSE108011 and GSE72624 respectively. The studies are published as Vaccari, M., et al., Adjuvant-dependent innate and adaptive immune signatures of risk of SIVmac251 acquisition, Nat. Med. 22, 762-770 (2016) and Vaccari, M., et al., HIV vaccine candidate activation of hypoxia and the inflammasome in CD14(+) monocytes is associated with a decreased risk of SIVmac251 acquisition, Nat. Med. 24, 847-856 (2018), respectively. For the DNA-SIV/ALVAC+gp120 study (N=12), microarray data from 1 week after the second boost time points were analyzed. For the ALVAC-SIV/gp120 study (N=27), time points 24 hours after the third immunization were analyzed. All microarray datasets used in this study were generated on the Human HT-12 v4.0 expression beadchip platform (Illumina). The samples in each study were grouped into outcomes after challenge or infection status post-vaccination at the end of 3 years in the study.

Immune Response Data

Functional immune response data for the Barouch I and Barouch II studies were described previously in those studies. Additional assays including IgG breadth and antigen-dependent neutrophil phagocytosis (ADNP) were analyzed. IgG binding activity was measured against 15 SIV antigens in the Barouch I study and 22 HIV antigens in the Barouch II study. IgG breadth was calculated by the sum of the measurements whose binding activity were above the median of the measurement across all animals in the trial. Antibody-dependent neutrophil-mediated phagocytosis (ADNP) assay was measured in the Barouch II study. ADNP was assessed by the measurement of the uptake of antibody-opsonized, antigen-coated fluorescent beads by primary neutrophils. Biotinylated A244 gp120 was used to saturate the binding sites on 1 μm fluorescent neutravidin beads (Invitrogen). Excess antigen was removed by washing the beads, which were then incubated with NHP antibody samples for 20 minutes at 37° C. Leukocytes were isolated from blood collected from HIV seronegative donors after ACK lysis of red blood cells. Following opsonization, the freshly isolated leukocytes were added, and the cells were incubated for 1 hour at 37° C. to allow phagocytosis. The cells were then stained for CD66b to identify neutrophils, fixed, and the extent of phagocytosis was measured by flow cytometry on a Stratedigm S1000EXi flow cytometer (San Jose, Calif.). The data are reported as a phagocytic score as described previously in Darrah, P. A. et al., Multifunctional TH1 cells define a correlate of vaccine-mediated protection against Leishmania major, Nat. Med. 13, 843-50 (2007).

Quantitative PCR

Gene expression data were confirmed by quantitative PCR (qPCR). Since only small amounts of RNA were available, the Fluidigm BioMark platform was used for validation (Fluidigm, San Francisco, Calif.). cDNA was synthesized by reverse transcription (RT) from 2.5 ng RNA extracted from sorted B cells using RT preamplification reaction mix, composed of TaqMan® Fast Virus 1-Step Master Mix (Applied Biosystems) and a pooled 96 target TaqMan® assay mix (ThermoFisher Scientific; Waltham, Mass.). The RT reaction was performed in three steps: cDNA synthesis (50° C. for 10 min), denaturation (95° C. for 2 min), and preamplification (18 cycles of 95° C. for 15 sec and 60° C. for 4 min).

The synthesized cDNA was diluted 1:5 with DNA Suspension Buffer (Teknova). TaqMan® Gene Expression assays (ThermoFisher Scientific) and sample mixes were added to a primed GE 96.96 Dynamic Array chip, which was loaded into the IFC Controller, and subsequently amplified on the BioMark HD using the GE 96×96 standard v2 protocol (all Fluidigm) per manufacturer's instructions. The cycle threshold (Ct) values were validated and exported using the Fluidigm BioMark gene expression real-time PCR analysis software (Fluidigm). Each target gene Ct value was normalized against a housekeeping gene, GAPDH. Individual gene expression was calculated relative to a healthy control NHP sample using the ΔΔCt method. All assays were previously validated for exponential amplification and linear dependence on input.

Statistical Analysis

To reduce the stringent limitations of differential expression analyses at the single gene level imposed by multiple hypothesis corrections, normalized RNA-Seq transcription profiles from CD4+ and CD8+ T-cells, B cells, and NK cells were analyzed using the Gene Set Enrichment Analysis (GSEA) method, as discussed in Subramanian, A., et al., Gene set enrichment analysis: a knowledge-based approach for interpreting genome-wide expression profiles, Proc. Natl. Acad. Sci. USA 102, 15545-15550 (2005). GSEA uses gene sets established a priori that have been grouped together based on involvement in particular biological functions or chromosomal location. Genes are ranked based on a signal-to-noise metric that differentiates enriched genes with user provided phenotypes. These genes are then evaluated to determine if a member of a gene set occurs at the top or bottom of the ranked list and generates a Kolmogorov-Smirnov-like statistic called Enrichment Score (ES). The statistical significance of the ES is estimated by gene-set permutation tests in order to produce a null distribution. Genes were analyzed against the C2 canonical pathways collections comprising 1329 individual gene sets from BioCarta, KEGG, Reactome, and other online databases, and the C7 immunologic signatures gene set collection comprising 4872 individual gene sets representing cell types, states, and perturbations within the immune system generated by manual curation of published studies. For each GSEA comparison, gene sets were tested to evaluate if the genes significantly associated with protection against challenge. Signatures were considered significantly protective if genes were enriched in the protected versus infected animals using a threshold of P<0.001 and Normalized Enrichment Score (NES) ≥1.4. Signatures were further down-selected if they overlapped in the Ad26/gp140 arms of the Barouch I and Barouch II studies.

One signature in B cells, having the GSEA name GSE29618_BCELL_VS_MONOCYTE_DAY7_FLU_VACCINE_DN, associated with protection against acquisition. This gene set previously expressed in humans contains 200 genes that are down-regulated in comparison to B cells from influenza vaccines at day 7 versus monocytes from influenza vaccines at day 7 and were published in Nakaya, H. I., et al., Systems biology of vaccination for seasonal influenza in humans, Nat. Immunol., 12(8), 786-95 (2011). Of the 200 genes in this gene set, 140 were expressed in the RNA-Seq data generated in the NHP samples from Barouch I and Barouch II discussed above.

For further analysis, a total of 36 enriched genes in the B cell signature from the Barouch I study were identified by GSEA and were selected for downstream processing as described below. The 36 enriched genes included the following: ACSL1, ALDH3B1, BHLHE40, CD302, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, OGFRL1, PHACTR2, RAB32, REEP4, RNF130, RRAGD, RTN1, SDCBP, SIRPA, SLC27A3, ST3GAL6, TKT, TNFRSF1B, TNFSF13, and VEGFA. An average gene enrichment scores (GES) for each sample calculated from the 36 enriched genes are shown below in Table 2.

TABLE 2
Average GES for each sample (N = 10) from Barouch I data set

| | | Average |
|---|---|---|
| | | GES + |
| | | constant to |
| Sample | Average | yield positive |
| ID No: | GES | value |
| 72-06 | 0.0096 | 1.7719 |
| (Infected) | | |
| 75-06 | −0.7623 | 1 |
| (Infected) | | |
| 87-06 | −0.5717 | 1.1905 |
| (Infected) | | |
| 90-06 | −0.3419 | 1.4203 |
| (Infected) | | |
| 302-07 | −0.1564 | 1.6059 |
| (Infected) | | |
| 69-06 | 0.4047 | 2.1670 |
| (Protected) | | |
| 136-06 | 0.6675 | 2.4398 |
| (Protected) | | |
| 190-07 | −0.3168 | 1.4455 |
| (Protected) | | |
| 191-07 | 0.7184 | 2.4807 |
| (Protected) | | |
| 280-07 | 0.3488 | 2.1111 |
| (Protected) | | |

Likewise, a total of 62 enriched genes in the B cell signature from the Ad26/Ad26+gp140 arm from the Barouch II study were identified by GSEA. The 62 enriched genes included the following: ACSL1, AHNAK, AOAH, ARHGEF10L, ARRB1, BHLHE40, CAMKK2, CD86, CD163, CDC42EP3, CEBPA, CEBPD, CLEC4A, CPD, CPPED1, CREB5, CREG1, CSF3R, CST1, CTNNA1, DAPK1, DOCK5, EMILIN2, ENO1, ERMAP, FAM105A, FBXL5, GLA, GNS, HBEGF, HEXB, HPSE, HSBP1, ID2, IRAK3, KIAA0513, LAMP2, LMNA, MAFB, MAPKAPK3, MICAL2, MYO1F, NFIL3, OGFRL1, PGD, PSAP, RAB27A, RNF130, RRAGD, RXRA, SCARB2, SDCBP, SEMA4A, SLC31A2, SLC36A1, STX11, TBC1D8, TNFRSF1B, TNFSF13, VEGFA, VPS37C, and WDFY3. Gene enrichment scores for the 62 enriched genes are shown below in Table 3.

TABLE 3
Average GES for each sample (N = 12) from Barouch
II data set, Ad26/Ad26 + gp140 arm

| | | Average |
|---|---|---|
| | | GES + |
| | | constant to |
| Sample | Average | yield positive |
| ID No: | GES | value |
| DEMN_54 | −0.0452 | 1.5440 |
| (Protected) | | |
| DEMP_54 | 0.0921 | 1.6813 |
| (Protected) | | |
| DENA_54 | −0.0417 | 1.5475 |
| (Protected) | | |
| DENB_54 | 0.0418 | 1.6309 |
| (Protected) | | |
| DENH_54 | 1.6135 | 3.2023 |
| (Protected) | | |
| DENJ_54 | 0.1280 | 1.7171 |
| (Protected) | | |
| DENL_54 | −0.1855 | 1.4037 |
| (Protected) | | |
| DFHE_54 | 0.2324 | 1.8216 |
| (Protected) | | |
| DEMX_54 | −0.4338 | 1.1553 |
| (Infected) | | |
| DEMZ_54 | −0.5655 | 1.0237 |
| (Infected) | | |
| DFFZ_54 | −0.5892 | 1 |
| (Infected) | | |
| MHD_54 | −0.2469 | 1.3423 |
| (Infected) | | |

Similarly, a total of 52 enriched genes in the B cell signature from the Ad26/gp140 arm from the Barouch II study were identified by GSEA. The 52 enriched genes included the following: ALDOA, AOAH, AP2S, APOBR, ARHGEF10L, ATF3, CD163, CEBPD, CLEC4A, CREB5, CSFIR, CSF3R, CST3, CTNNA1, DOCK5, DUSP6, EFHD2, ERMAP, FCGRT, GAA, GABARAPL1, GNAO, GRN, H2AFY, HBEGF, HSBP1, ICAM1, IRAK3, IL17RA, KIAA0513, KLF10, LST1, MAFB, MCL1, NAGA, NAMPT, NLRP3, OGFRL1, PSAP, PSTPIP1, RAB32, RGS10, RNF130, SIRPA, SLCO3A1, TBC1D8, TKT, TNFSF13, TRIB1, TYROBP, VIM, and WDFY3. Gene enrichment scores for the 52 enriched genes are shown below in Table 4.

TABLE 4
Average GES for each sample (N = 11)
from the Barouch II data set, Ad26/gp140 arm

| | | Average |
|---|---|---|
| | | GES + |
| | | constant to |
| Sample | Average | yield positive |
| ID No: | GES | value |
| DEKG_54 | 0.3322 | 2.0020 |
| (Protected) | | |
| DEM5_54 | 1.1984 | 2.8683 |
| (Protected) | | |
| DFBX_54 | −0.0675 | 1.6024 |
| (Protected) | | |
| DFCJ_54 | 0.9152 | 2.5850 |
| (Protected) | | |
| DEIL_54 | −0.3769 | 1.2930 |
| (Infected) | | |
| DEJA_54 | −0.3581 | 1.3118 |
| (Infected) | | |
| DEJE_54 | −0.6698 | 1 |
| (Infected) | | |
| DEJG_54 | −0.3489 | 1.3209 |
| (Infected) | | |
| DEKF_54 | −0.2564 | 1.4135 |
| (Infected) | | |
| DELM_54 | −0.5534 | 1.1164 |
| (Infected) | | |
| MH3_54 | −0.1852 | 1.8550 |
| (Infected) | | |

A composite GES of the enriched genes in the Barouch I study was computed by averaging Z scores of the normalized gene expression for each monkey. Prediction models for infection status were generated using the composite GES from 32 of the 36 enriched genes from Barouch I. Four of the enriched genes, including ALDH3B1, CD302, RTN1 and SLC27A3, were not considered as they did not pass the EdgeR filtering step. Accordingly, the following 32 genes were used to compute the GES: ACSL1, BHLHE40, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, OGFRL1, PHACTR2, RAB32, REEP4, RNF130, RRAGD, SDCBP, SIRPA, ST3GAL6, TKT, TNFRSF1B, TNFSF13, and VEGFA.

A GES was also computed for the same 32 genes in the two different arms of the Barouch II study (i.e., the Ad26/Ad26+gp140 arm and the Ad26/gp140 arm). Bootstrap optimism-corrected Area under Receiver Operating Characteristics (ROC) Curves (AUC) were used for internal validation of model discrimination. The performance of the classifier was assessed using AUC. An AUC of 0.5 indicated random discrimination and 1 indicated perfect discrimination capabilities. The performance of this classifier was then assessed on the Ad26/gp140 test and Ad26/Ad26+gp140 validation datasets of the Barouch II study. Similar prediction analysis was performed using a GES of 30 enriched genes from the Barouch I study, and tested in the Ad26/MVA+gp140 arm in the Barouch II study, as two of the enriched genes (TKT, RRAGD) did not pass the filtering step.

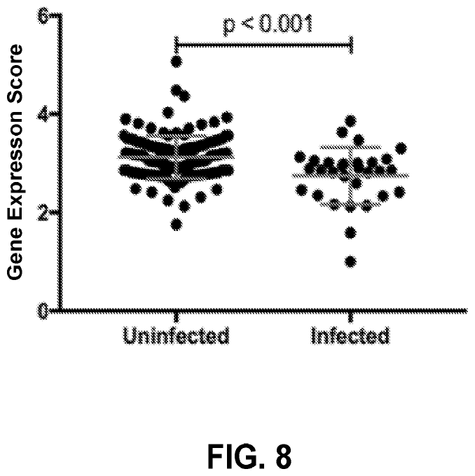

For the RV144 human clinical study, discussed below in Example 2, a total of 63 enriched genes were identified by GSEA. The 63 enriched genes included the following: ADAM9, ALDH3B1, AMPD2, AP2SL, ARRB1, C5AR1, CAMKK2, CCR1, CD14, CD163, CD68, CEBPA, CSFIR, CST3, CTBP2, CTSD, DAPK1, DUSP6, FCGRT, FLVCR2, FZDJ, GAA, GAS7, GRN, HMOX1, IL17RA, IMPA2, KIAA0513, LEPROT LGALS2, LMNA, LRPAP1, MAFB, MPP1, MYO1F, NAGA, NPL, PGD, PKM2, PSAP, PTGER2, PAN, PYGL, QPCT, RAB20, RBM47, RNF130, SCARB2, SEMA4A, SERINC5, SIRPA, SIRPB1, SLC36A1, SLCO3A1, SORT1, SULT1A2, TBC1D8, TBXAS1, TIMP2, TKT, TYROBP, ZDHHC7, and ZNF467. The mean GES is statistically significantly greater in subjects who received HIV vaccine, developed protective immunity, and remained uninfected (3.11), than in those who received HIV vaccine but acquired infection (2.73) (p<0.001) (FIG. 8).

Protection as defined by number of challenges required for infection was analyzed by Cox proportional hazard regression using the GES as a continuous variable for both the Barouch I and Barouch II studies. The GES from the two arms of the Barouch II study (Ad26/gp140 and Ad26/Ad26+gp140) were combined for this analysis and all other analysis mentioned below.

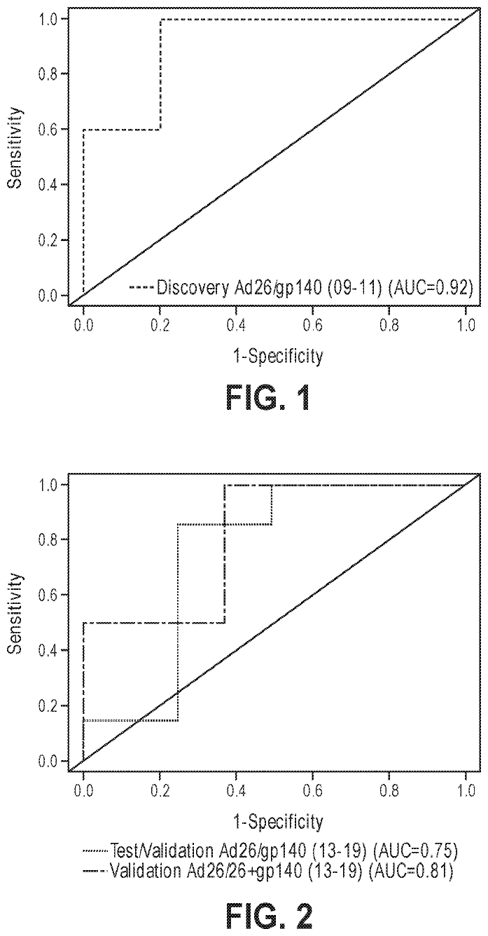

As shown in FIG. 1, the ROC curve (AUC 0.92) illustrates the ability of the GES of enriched genes from the Barouch I study to predict protection from virus acquisition. For the Barouch I samples, a hazard ratio of 0.01 was calculated, with a 95% confidence interval (CI) of 0.0002-0.68 and a p-value of 0.032. Likewise, FIG. 2 shows the ROC curves (AUC 0.75; AUC 0.81) for both arms of the Barouch II study, demonstrating the ability of the GES of the enriched genes to predict protection from virus acquisition. For the Barouch II samples, a hazard ratio of 0.05 was calculated, with a 95% CI of 0.004-0.58 and a p-value of 0.017.

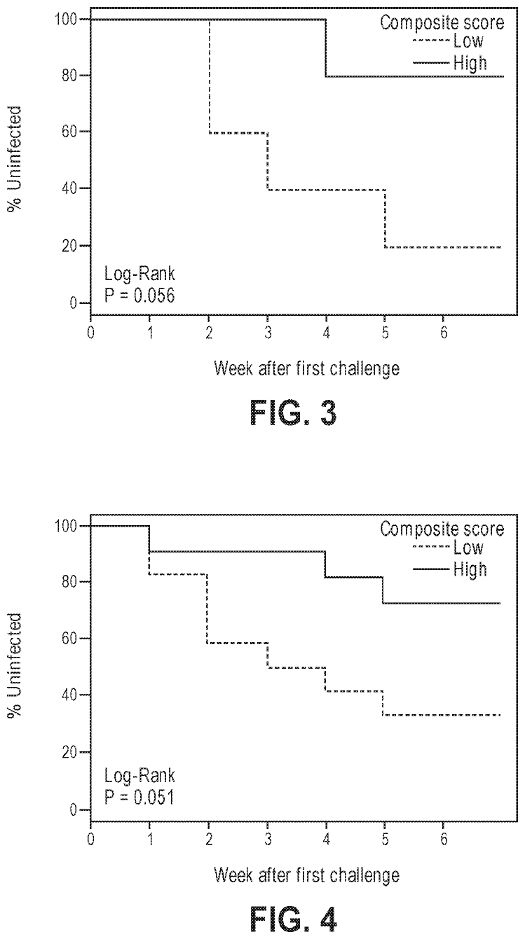

GES was also tested as a categorical variable by stratifying into low versus high groups as determined by the median. All uninfected animals were censored at the last challenge. Survival curves were estimated using the Kaplan-Meier method. As shown in FIG. 3, the composite GES as a categorical variable of the high group in the Barouch I study were higher than those of the low group (p-value=0.056). When the composite GES was calculated as a continuous variable, the results were significantly different (hazard ratio=0.10; 95% CI=0.01-0.82; p-value=0.03), demonstrating reduced risk of infection. FIG. 4 likewise demonstrates a reduced risk of infection in the composite GES as a categorical variable of the high group in the Barouch II study as compared to those in the low group (p-value=0.051). When GES was calculated as a continuous variable for this group, the results were significantly different (hazard ratio=0.32; 95% CI=0.12-0.81; p-value=0.02).

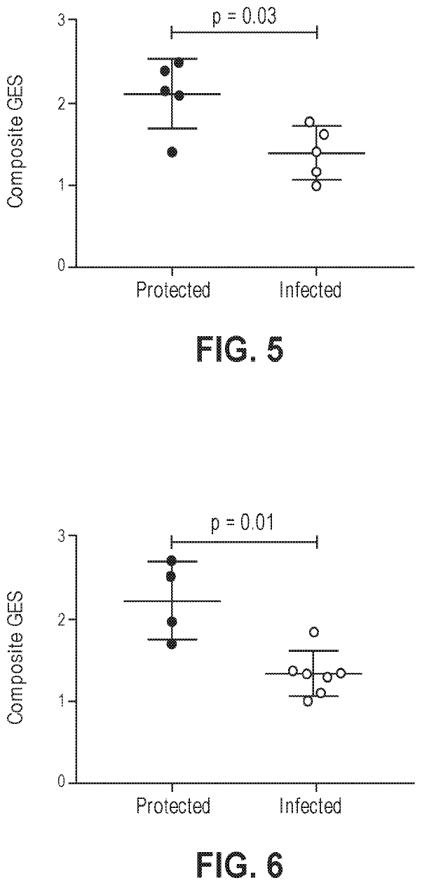

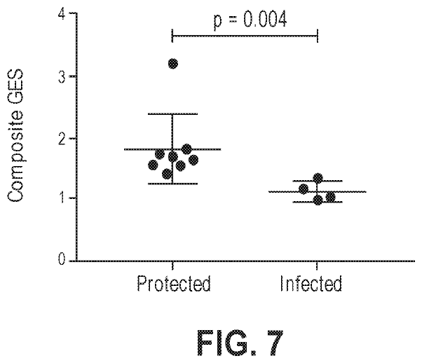

FIG. 5-7 are scatter plot graphs showing the composite GES calculated for differentially expressed gene sets in both protected and infected groups of various study populations. As shown in FIG. 5, the GES, comprising 32 differentially expressed genes, was significantly higher (p=0.03) in the protected group as compared to the infected group in the Barouch I study population. As shown in FIG. 6, the GES, comprising 52 differentially expressed genes, is significantly higher (p=0.01) in the protected group as compared to the infected group in the Ad26/gp140 arm of the Barouch II study population, and FIG. 7 illustrates that the GES, comprising 62 differentially expressed genes, is significantly higher (p=0.004) in the protected group as compared to the infected group in the Ad26/Ad26+gp140 arm of the Barouch II study population.

A two-sided P value of less than 0.05 was considered statistically significant for all statistical analysis described above. All descriptive and inferential statistical analyses were performed using R 3.4.1 GUI 1.70 build (7375) v3.0 and higher, and GraphPad Prism 7 statistical software packages (GraphPad Software, La Jolla, Calif.).

### Example 2. The Association of the GES is a Stronger Correlate of Protection than the V1V2-Specific IgG Antibodies Study

RV144 Human Clinical Study

RV144 is a phase III efficacy trial in which a ‘prime-boost’ vaccine strategy is evaluated for prevention of infection and amelioration of disease course. The purpose of this study is to determine whether immunizations with an integrated combination of ALVAC-HIV (vCP1521) boosted by AIDSVAX gp120 B/E preventHIV infection in healthy Thai volunteers. ALVAC-HIV (vCP1521) from Sanofi Pasteur is given as the ‘prime’ vaccine at months 0, 1, 3 and 6; AIDSVAX gp120 B/E from VaxGen is given as the ‘boost’ at months 3 and 6. This regimen is given to 8,000 adult Thai subjects, while another 8,000 receive placebos in a double-blinded, randomized manner. Following the completion of each subjects' immunization phase, he/she is followed for 3 years with clinic visits every 6 months with HIV testing and pre- and post-test counseling. Subjects who become HIV infected are counseled, referred to HIV treatment facilities for management according to national guidelines, and offered enrollment in a protocol for extended follow-up.

Binding ELISA to Scaffolded HIV-1 Envelope V1V2

Haynes, et al., Immune-Correlates Analysis of an HIV-1 Vaccine Efficacy Trial, New England J. Med. 2012; 366:1275-1286, disclosed an correlation between conferred immunity in the RV144 study and increased binding of IgG antibodies to variable regions 1 and 1 (V1V2) of HIV-1 envelope proteins. In the procedure disclosed in Haynes et al. 2012, Immulon 4HBX plates (Thermo Scientific) were coated with 1 g/ml scaffolded V1V2 (murine leukemia virus gp70-V1V2), stored overnight at 4° C. and then washed 6 times with PBS containing 0.05% Tween-20, pH 7.4, before incubation for 1.5 h at 37° C. with RV144 plasma diluted 1:100 in 15% RPMI media. The plates were washed again 6 times. For detection of bound V1V2 antibodies, alkaline phosphatase-conjugated goat anti-human IgG (SouthemBiotech 1:2000) was added for 1.5 h at 37° C. After washing, 10% diethanolamine substrate was added for 30 min at room temperature to develop color, and the plates were read at 405 nm. At each step, every well contained 50 μl. Each experiment included four negative control wells which contained 15% media without plasma. Each case-control specimen was run in duplicate in each experiment, and three experiments were performed. See Haynes et al. 2012 at Supplementary Appendix. Haynes concluded that “the binding of IgG antibodies to . . . V1V2 of HIV-1 envelope proteins (Env) correlated inversely with the rate of HIV-1 infection (estimated odds ratio, 0.57 per 1-SD increase; P=0.02; q=0.08).” Haynes et al. 2012 at Abstract.

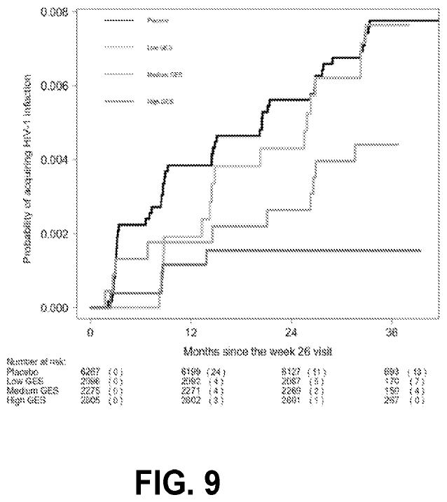

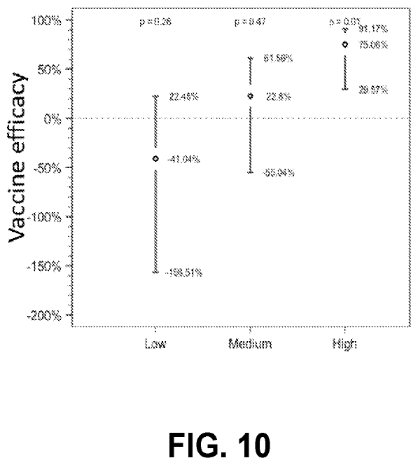

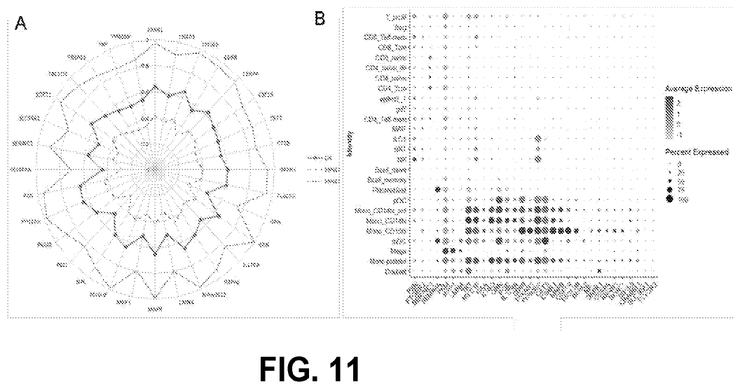

The association of the GES in the RV144 study, however, is a stronger correlate of protection than the V1V2-specific IgG antibodies study (Haynes et al. 2012) and associates with reduced risk of HIV acquisition and increased vaccine efficacy (Table 5). Vaccinated individuals with high (i.e., top one third of all GES scores) GES have a reduced probability of acquiring HIV-1 acquisition and increased vaccine efficacy (N=170) (FIG. 9, FIG. 10), versus individuals with a medium (i.e., middle one third) GES or a low (i.e., bottom one third) GES. A subset of genes from the gene signature also associated with decreased risk of acquisition in a univariate analysis (Odds ratio<1.0, P<0.05, q<0.1) (FIG. 11A). A stepwise logistic regression multivariate analysis identified specific genes including SEMA4A, SLC36A1, SERINC5, IL17RA, CTSD, CD68, and GAA with reduced risk of acquisition (FIG. 11A). Single cell RNA-Seq (scRNA-seq) data from peripheral blood reveal that majority of the genes in the signature are expressed in cells of the myeloid lineage (FIG. 11B). These genes have a number of biological functions including phagocytosis, immune regulation and proinflammation. Hence, this signature bearing the hallmark of genes that are normally expressed in monocytes is associated with increased HIV vaccine efficacy and immunogenicity. The protective genes did not overlap with the enriched genes in the Barouch I and Barouch II studies.

TABLE 5
GES is associated with lower odds of HIV acquisition
compared to Env-specific IgG antibodies.

| RV144 | N | Odds Ratio | 95% CI | P value | Reference |
|---|---|---|---|---|---|
| GES | 170 | 0.37 | 0.22-0.63 | 0.0002 | |
| Gp70-V1V2 | 246 | 0.7 | 0.49-1.02 | 0.06 | Haynes |
| (IgG) | | | | | et al. |
| | | | | | 2012 |

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims. The claims are intended to cover the components and steps in any sequence which is effective to meet the objectives there intended, unless the context specifically indicates the contrary.

What is claimed is:

1. A method for predicting effectiveness of a human immunodeficiency virus (HIV) or a simian immunodeficiency virus (SIV) vaccine in a subject that has been vaccinated with the HIV or the SIV vaccine, the method comprising:
(a) detecting expression levels of multiple genes and/or gene products in a biological sample from the subject, wherein the multiple genes and/or gene products have been identified as a differentially expressed gene set associated with efficacy of the HIV or SIV vaccine;
(b) calculating a first composite gene expression score (GES) for the differentially expressed gene set in the biological sample from the subject; and
(c) calculating an average second composite GES for the differentially expressed gene set in biological samples from a plurality of subjects that have been vaccinated with the HIV or SIV vaccine and are not immune to HIV or SIV,
wherein a first composite GES that is greater than the average second composite GES indicates effectiveness of the HIV or SIV vaccine.

2. A method for predicting protective immunity to HIV or SIV infection in a subject that has been vaccinated with an HIV or SIV vaccine, the method comprising:
(a) detecting expression levels of multiple genes and/or gene products in a biological sample from the subject, wherein the multiple genes and/or gene products have been identified as a differentially expressed gene set associated with protective immunity against HIV or SIV;
(b) calculating a first composite gene expression score (GES) for the differentially expressed gene set in the biological sample from the subject; and
(c) calculating an average second composite GES for the differentially expressed gene set in biological samples from a plurality of subjects that have been vaccinated with the HIV or SIV vaccine and are not immune to HIV or SIV,
wherein a first composite GES that is greater than the average second composite GES indicates the subject that has been vaccinated with the HIV or SIV vaccine has protective immunity against HIV or SIV.

3. The method of any one of claims 1-2, wherein the HIV or SIV vaccine comprises an adenovirus serotype 26 (Ad26) vector-based vaccine, a canarypox virus vector-based vaccine, or a retrovirus vector-based vaccine.

4. The method of any of the preceding claims, wherein the HIV or SIV vaccine comprises an HIV or SIV envelope glycoprotein.

5. The method of any of the preceding claims, further comprising a step of identifying the differentially expressed gene set associated with vaccine effectiveness and protective immunity against HIV or SIV.

6. The method of any one of the preceding claims, wherein the subject comprises humans and non-human animals.

7. The method of any one of the preceding claims, wherein the subject is a human.

8. The method of any one of the claims 1-6, wherein the subject is a non-human animal.

9. The method of claim 8, wherein the non-human animal is a non-human primate.

10. The method of any of the preceding claims, wherein the differentially expressed gene set is associated with a B-cell gene signature or a monocyte gene signature.

11. The method of any of the preceding claims, wherein the biological sample comprises blood, peripheral blood mononuclear cells, white blood cells, lymphocytes, granulocytes, monocytes, macrophages, or any combination thereof.

12. The method of any of the preceding claims, wherein the first and second composite GES are calculated by averaging Z scores calculated by comparison to normalized gene expression for each gene in the differentially expressed gene set.

13. The method of claim 12, wherein the normalized gene expression is measured from biological samples from subjects that have not been vaccinated with the HIV or SIV vaccine and are not infected with HIV or SIV.

14. The method of any of the preceding claims, wherein the differentially expressed gene set comprises at least 3, at least 10, at least 15, at least 20, at least 25, at least 30, or 32 genes selected from the group consisting of ACSL1, BHLHE40, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, OGFRL1, PHACTR2, RAB32, REEP4, RNF130, RRAGD, SDCBP, SIRPA, ST3GAL6, TKT, TNFRSF1B, TNFSF13, and VEGFA.

15. The method of claim any of the preceding claims, wherein the differentially expressed gene set comprises BHLHE40, OGFRL1, and TNFSF13.

16. The method of any of the claims 1-13, wherein the differentially expressed gene set comprises at least 7, at least 10, at least 20, at least 30, at least 40, at least 50, at least 60, or 63 genes selected from the group consisting of ADAM9, ALDH3B1, AMPD2, AP2S1, ARRB1, C5AR1, CAMKK2, CCR1, CD14, CD163, CD68, CEBPA, CSFIR, CST3, CTBP2, CTSD, DAPK1, DUSP6, FCGRT, FLVCR2, FZD1, GAA, GAS7, GRN, HMOX1, IL17RA, IMPA2, KIAA0513, LEPROT, LGALS2, LMNA, LRPAP1, MAFB, MPP1, MYO1F, NAGA, NPL, PGD, PKM2, PSAP, PTGER2, PXN, PYGL, QPCT, RAB20, RBM47, RNF130, SCARB2, SEMA4A, SERINC5, SIRPA, SIRPB1, SLC36A1, SLCO3A1, SORT1, SULT1A2, TBC1D8, TBXAS1, TIMP2, TKT, TYROBP, ZDHHC7, and ZNF467.

17. The method of claim 16, wherein the differentially expressed gene set comprises SEMA4A, SLC36A1, SERINC5, IL17RA, CTSD, CD68, and GAA.

18. The method of any of the preceding claims, wherein the composite GES is calculated based on measuring or detecting levels of mRNA, cDNA, or protein products of the genes in the differentially expressed gene set in the biological sample.

19. A kit for use in predicting effectiveness of an HIV or SIV vaccine in a subject, the kit comprising:
(a) reagents for measuring or detecting expression levels of genes and/or gene products selected from the group consisting of BHLHE40, OGFRL1, TNFSF13 and at least 5, at least 10, at least 15, at least 20, at least 25, or 29 genes selected from the group consisting of ACSL1, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, PHACTR2, RAB32, REEP4, RNF130, RRAGD, SDCBP, SIRPA, ST3GAL6, TKT, TNFRSF1B, and VEGFA; and
(b) instructions for how to use the kit.

20. A kit for use in predicting effectiveness of HIV or SIV vaccination in a subject, the kit comprising:
(a) reagents for measuring or detecting expression levels of genes and/or gene products selected from the group consisting of SEMA4A, SLC36A1, SERINC5, IL17RA, CTSD, CD68, GAA and at least 5, at least 10, at least 20, at least 30, at least 40, at least 50, or 56 genes selected from the group consisting of ADAM9, ALDH3B1, AMPD2, AP2S1, ARRB1, C5AR1, CAMKK2, CCR1, CD14, CD163, CEBPA, CSFIR, CST3, CTBP2, DAPK1, DUSP6, FCGRT, FLVCR2, FZD1, GAS7, GRN, HMOX1, IMPA2, KIAA0513, LEPROT, LGALS2, LMNA, LRPAP1, MAFB, MPP1, MYO1F, NAGA, NPL, PGD, PKM2, PSAP, PTGER2, PXN, PYGL, QPCT, RAB20, RBM47, RNF130, SCARB2, SIRPA, SIRPB1, SLCO3A1, SORT1, SULT1A2, TBC1D8, TBXAS1, TIMP2, TKT, TYROBP, ZDHHC7, and ZNF467; and
(b) instructions for how to use the kit.

21. A kit for use in predicting protective immunity to HIV or SIV infection in a subject that has been vaccinated with an HIV or SIV vaccine, the kit comprising:
(a) reagents for measuring or detecting expression levels of genes and/or gene products selected from the group consisting of BHLHE40, OGFRL1, TNFSF13 and at least 5, at least 10, at least 15, at least 20, at least 25, or 29 genes selected from the group consisting of ACSL1, CD4, CDC42EP3, CDC42EP4, CREB5, CREG1, DAPK1, DOCK5, EMILIN2, ERMAP, GAS7, GLA, IRAK3, LAMP2, LMNA, MGST2, NFIL3, NLRP3, PHACTR2, RAB32, REEP4, RNF130, RRAGD, SDCBP, SIRPA, ST3GAL6, TKT, TNFRSF1B, and VEGFA; and
(b) instructions for how to use the kit.

22. A kit for use in predicting protective immunity to HIV or SIV infection in a subject that has been vaccinated with an HIV or SIV vaccine, the kit comprising:
(a) reagents for measuring or detecting expression levels of genes and/or gene products selected from the group consisting of SEMA4A, SLC36A1, SERINC5, IL17RA, CTSD, CD68, GAA and at least 5, at least 10, at least 20, at least 30, at least 40, at least 50, or 56 genes selected from the group consisting of ADAM9, ALDH3B1, AMPD2, AP2S1, ARRB1, C5AR1, CAMKK2, CCR1, CD14, CD163, CEBPA, CSFIR, CST3, CTBP2, DAPK1, DUSP6, FCGRT, FLVCR2, FZD1, GAS7, GRN, HMOX1, IMPA2, KIAA0513, LEPROT, LGALS2, LMNA, LRPAP1, MAFB, MPP1, MYO1F, NAGA, NPL, PGD, PKM2, PSAP, PTGER2, PXN, PYGL, QPCT, RAB20, RBM47, RNF130, SCARB2, SIRPA, SIRPB1, SLCO3A1, SORT1, SULT1A2, TBC1D8, TBXAS1, TIMP2, TKT, TYROBP, ZDHHC7, and ZNF467; and
(b) instructions for how to use the kit.
